(19) United States
(12) Patent Application Publication
(10) Pub. No.: US 2023/0193368 A1
(43) Pub. Date: Jun. 22, 2023

(54) CRISPR/CAS CHAIN REACTION SYSTEMS AND METHODS FOR AMPLIFYING THE DETECTION SENSITIVITY OF CRISPR-BASED TARGET DETECTION

(71) Applicant: UNIVERSITY OF FLORIDA RESEARCH FOUNDATION, Gainesville, FL (US)

(72) Inventors: Santosh R. RANANAWARE, Gainesville, FL (US); Piyush K. JAIN, Gainesville, FL (US); Long T. NGUYEN, Gainesville, FL (US); Marco A. DOWNING, Gainesville, FL (US)

(21) Appl. No.: 17/928,400

(22) PCT Filed: May 28, 2021
(86) PCT No.: PCT/US2021/034971

§ 371 Date: Nov. 29, 2022

Related U.S. Application Data
(60) Provisional application No. 63/032,370, filed on May 29, 2020.
(60) Provisional application No. 63/191,890, filed on May 21, 2021.

(51) Int. Cl. C12Q 1/6848

(52) U.S. Cl. CPC C12Q 1/6848; C12Q 2600/16; C12Q 1/701

(57) ABSTRACT

The present disclosure provides CRISPR/Cas chain reaction (CCR) systems and methods for amplifying the detection sensitivity of a primary CRISPR-based target detection (CBTD) system for detecting targets. Also described are methods of using CCR systems to amplify the detection sensitivity of primary CBTD systems to detect a target without a target preamplification step. Multiplexed CBTD systems for detecting a target using two different Cas enzyme systems are provided.

### RELATED APPLICATION

This application claims the benefit of U.S. Provisional Application Ser. No. 63/032,370, titled “Systems and Methods for Amplified CRISPR/Cas-based Detection,” filed May 29, 2020 and U.S. Provisional Application Ser. No. 63/191,890, having the same title and filed May 21, 2021. Each of these applications is incorporated herein by reference in their entirety.

### FIELD

The present disclosure relates to CRISPR/Cas complex-based systems and methods.

### BACKGROUND

The development of CRISPR/Cas systems for rapid, point-of-care detection of nucleic acid targets for diagnosing diseases, such as cancer and viruses, has increased recently. The ongoing SARS-CoV-2 pandemic has vastly underscored the need for developing rapid, accurate and sensitive techniques for pathogen detection. Contemporary diagnostic methods that are based on reverse transcriptase polymerase chain reaction (RT-qPCR) are widely used, but are handicapped by their dependency on expensive reagents, sophisticated equipment, and trained personnel. CRISPR-Cas systems have emerged as a widely adopted diagnostic tool for the detection of SARS-CoV-2 and other viruses and conditions within the past year.

Class 2 type V and VI single effector Cas proteins, such as Cas12a and Cas13a, have been employed for the development of rapid, sensitive, and cost-effective detection platforms including DETECTR and SHERLOCK (Gootenberg et al., Science, 2017; Gootenberg et al., Science, 2018; Chen et al., Science, 2018; Brougton et al, Nat. Biotechnol., 2020; Young et al, NEJM., 2020) due to their robust trans-cleavage activity. The Cas12a-based DETECTR technology from Mammoth Biosciences and Cas13a-based SHERLOCK technology from Sherlock Biosciences are two CRISPR-based detection systems that are now approved by the FDA under EUA as lab-based diagnostics for detecting SARS-CoV-2 RNA. These platforms combine nucleic acid pre-amplification methods, such as RT-LAMP, RT-RPA, RT-HDA and other isothermal amplification steps, with the trans-cleavage ability of Type V and Type VI Cas effectors, for specific recognition of nucleic acid targets.

However, CRISPR-based detection methods suffer from a low detection sensitivity at room temperature without a target pre-amplification step. Although several testing technologies have received an FDA EUA or are under development, there is still an urgent need for the development of a multiplexable, rapid, sensitive, specific, inexpensive, easy-to-use, and accessible testing kit for SARS-CoV-2 and other viruses, infectious agents, other diseases and conditions (such as cancer and genetic disorders), that can be implemented as a point-of-care diagnostic and/or a home-based testing kit, as well as environmental monitoring devices and testing devices for food and agricultural products.

### SUMMARY

According to various aspects, the present disclosure provides CRISPR/Cas chain reaction (CCR) systems and methods for amplifying the detection of a target by primary CRISPR-based target detection as well as kits including the CCR systems of the present disclosure. The CCR systems of the present disclosure are universal and can be adapted for use with any primary CRISPR-based target detection system.

Embodiments of CCR systems of the present disclosure are for amplifying the detection sensitivity of a primary CRISPR-based target detection (CBTD) system, where the primary CBTD system includes a plurality of CRISPR-associated (Cas) enzymes with activatable trans-cleavage activity and a plurality of primary CRISPR RNAs (crRNA) capable of forming a complex with one of the Cas enzymes to form a primary crRNA/Cas complex, each primary crRNA having a primary guide sequence configured to bind a target, such that binding of the primary crRNA/Cas complex to the target activates the trans-cleavage activity of the Cas enzyme to produce an activated primary crRNA/Cas complex. According to various aspects the CCR systems of the present disclosure include: a plurality of secondary crRNAs capable of forming a complex with one of the Cas enzymes to form a secondary crRNA/Cas complex, each secondary crRNA comprising a secondary guide sequence configured to bind an activator; a plurality of activators, each activator comprising an oligonucleotide element complementary to and configured to bind the secondary crRNA, such that binding of the secondary crRNA/Cas complex to the activator activates the trans-cleavage activity of the Cas enzyme to produce an activated secondary crRNA/Cas complex; a plurality of blocking moieties bound either to the secondary crRNA or to the activator such that the bound blocking moiety prevents binding of the secondary crRNA to the Cas enzyme or prevents binding of the secondary crRNA/Cas complex to the activator, each blocking moiety including a cleavable sequence configured to be cleaved by an activated Cas enzyme of an activated primary crRNA/Cas complex or an activated secondary crRNA/Cas complex, such that cleavage of the cleavable sequence of the blocking moiety releases the blocking moiety allowing the secondary crRNA to bind another of the Cas enzymes and an activator to produce an activated secondary crRNA/Cas complex, resulting in a CRISPR/Cas chain reaction that produces additional activated secondary crRNA/Cas complexes; and a plurality of probes, each probe comprising an oligonucleotide element labeled with a detectable label, wherein the probe is configured to be cleaved by any of the activated Cas enzymes of the activated primary crRNA/Cas complexes or the activated secondary crRNA/Cas complexes to generate a detectable signal or a detectable molecule, wherein the CCR system amplifies the detection sensitivity compared to the CRISPR-based target detection (CBTD) system alone.

Some embodiments of the present disclosure include a CCR system for amplifying a primary CBTD system described above, where the CCR system includes: a plurality of secondary crRNAs capable of forming a complex with one of the Cas enzymes to form a secondary crRNA/Cas complex, each secondary crRNA having a secondary guide sequence configured to bind an activator; a blocking nucleotide sequence bound to the secondary crRNA that prevents binding of the secondary crRNA to the Cas enzyme or the activator, where the blocking nucleotide sequence includes one or more complementary segments bound to the secondary crRNA and one or more non-complementary, unbound segment having a sequence cleavable by an activated Cas enzyme of an activated primary crRNA/Cas complex or an activated secondary crRNA/Cas complex, such that cleavage of the one or more non-complementary segments of the blocking nucleotide releases the secondary crRNA from the blocking nucleotide such that the secondary crRNA can bind the activator and another of the Cas enzymes; a plurality of activators, each activator having an oligonucleotide element complementary to and configured to bind the secondary crRNA, such that binding of the secondary crRNA/Cas complex to the activator activates the trans-cleavage activity of the Cas enzyme to produce an activated secondary crRNA/Cas complex, resulting in a CRISPR/Cas chain reaction that produces additional activated secondary crRNA/Cas complexes; and a plurality of probes, each probe having an oligonucleotide element labeled with a detectable label, wherein the probe is configured to be cleaved by the activated Cas enzymes of the activated primary crRNA/Cas complexes or the activated secondary crRNA/Cas complexes to generate a detectable signal or a detectable molecule, wherein the CCR system amplifies the detection sensitivity compared to the CRISPR-based target detection (CBTD) system alone.

According to some aspects, the present disclosure also provides kits including the CCR systems of the present disclosure and a primary CBTD system such as described above.

The present disclosure also provides methods of amplifying the detection sensitivity of a primary CRISPR-based target detection (CBTD) system. Methods of the present disclosure can include combining the primary CBTD system with a sample including a target to be detected and a CRISPR/Cas chain reaction (CCR) system of the present disclosure, where the signal generated by the CCR system is greater than a signal produced from the primary CBTD system alone in the same amount of time.

According to some other aspects of the present disclosure, multiplexed CRISPR-based target detection (CBTD) systems are also provided. Such multiplexed CBTD systems can include: a primary CBTD system that includes: a plurality of primary Cas enzymes with activatable trans-cleavage activity and a plurality of primary crRNA capable of forming a complex with one of the primary Cas enzymes to form a primary crRNA/Cas complex, each primary crRNA comprising a primary guide sequence configured to bind a target, such that binding of the primary crRNA/Cas complex to the target activates the trans-cleavage activity of the primary Cas enzyme to produce an activated primary crRNA/Cas complex. The multiplexed CBTD systems also include a secondary CBTD system that includes: a plurality of secondary Cas enzymes with activatable trans cleavage activity, wherein the secondary Cas enzyme is different from the primary Cas enzyme; a plurality of secondary crRNAs capable of forming a complex with a secondary Cas enzyme to form a secondary crRNA/Cas complex, each having a secondary guide sequence configured to bind an activator; a plurality of activators, each activator having an oligonucleotide element complementary to and configured to bind the secondary crRNA, such that binding of the secondary crRNA/Cas complex to the activator activates the trans-cleavage activity of the secondary Cas enzyme to produce an activated secondary crRNA/Cas complex; a plurality of blocking moieties bound either to the secondary crRNA or to the activator such that the bound blocking moiety prevents binding of the secondary crRNA to the secondary Cas enzyme or prevents binding of the secondary crRNA/Cas complex to the activator, each blocking moiety having a cleavable sequence configured to be cleaved by an activated Cas enzyme of an activated primary crRNA/Cas complex, such that cleavage of the cleavable sequence of the blocking moiety releases the blocking moiety allowing the secondary crRNA to bind a secondary Cas enzymes and an activator to produce an activated secondary crRNA/Cas complex; and a plurality of probes, each probe having an oligonucleotide element labeled with a detectable label, wherein the probe is configured to be cleaved by the activated secondary crRNA/Cas complex to generate a detectable signal or a detectable molecule.

Other systems, methods, features, and advantages of the present disclosure will be or will become apparent to one with skill in the art upon examination of the following drawings and detailed description. It is intended that all such additional systems, methods, features, and advantages be included within this description, and be within the scope of the present disclosure.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates a schematic of a CCR system/method of the present disclosure.

FIGS. 2A-2C schematically illustrate various steps of an embodiment of a diagnostic test using the CCR methods/system of the present disclosure, as well as additional steps, such as obtaining and processing the sample and/or using the system in conjunction with a lateral flow assay, such as for point-of-care diagnostics.

FIG. 3 is a schematic illustration of a self-amplifying CCR system of the present disclosure for amplified DNA/RNA detection sensitivity that utilizes an amplifying secondary crRNA (“AMP:crRNA 3′7DNA-20PS) with its corresponding amplifying DNA target or activator “Amp DNA”.

FIGS. 4A-4E illustrate different design variations for blocked secondary crRNA according to embodiments of the present disclosure, including phosphorothiolated crRNA with cleavable DNA or RNA linker or extra-long DNA or RNA linker (FIG. 4A), a phosphorothiolated-crRNA with a cleavable linker or long DNA or RNA linker that forms a toe-hold loop (FIG. 4B), a crRNA with sterically blocking molecule attached via a cleavable DNA or RNA linker (FIG. 4C), a crRNA that forms a toe-hold loop with itself until cleaved at a DNA or RNA linker (FIG. 4D), or a surface locked crRNA coupled to a surface via a DNA or RNA linker (FIG. 4E).

FIG. 5 illustrates 5 models (A-E) of variations of modified crRNAs with unlinked DNA (or RNA) blocking moieties (uDNAs).

FIGS. 6A and 6B illustrate blocking of CRISPR/Cas activity by uDNAs of FIG. 5 having lengths ranging from 14 nt-41 nt. FIG. 6A illustrates mean RFU vs time for n=3 replicates, and FIG. 6B illustrates a heat map of mean RFU.

FIGS. 7A-7E are bar graphs illustrating HIV detection with CCR using blocked crRNA blocked by uDNA blockers of various lengths: uDNA14 (FIG. 7A), uDNA-21 (FIG. 7B), uDNA-28 (FIG. 7C), uDNA-41 (FIG. 7D), and uDNA-35 (FIG. 7E).

FIG. 8 illustrates 5 models (A-E) of variations of modified crRNAs with hairpin linked DNA (or RNA) blocking moieties.

FIGS. 9A and 9B illustrate blocking of CRISPR/Cas activity using hairpin modified blocked crRNAs illustrated in FIG. 8. FIG. 9A illustrates mean RFU vs time for n=3 replicates, and FIG. 9B illustrates a heat map of mean RFU.

FIGS. 10A and 10B illustrate detection of a synthetic SARS-CoV-2 target using a self-blocking hairpin modified crRNA that targets a short double-stranded GFP activator. FIG. 10A is a graph of mean RFU vs time for n=2 replicates, and FIG. 10B illustrates a heat map of mean RFU.

FIGS. 11A and 11B are bar graphs illustrating detection of a synthetic DNA target resembling SARS-CoV-2 (CoV) (FIG. 11A) or HIV genes (FIG. 11B) with each of 5 hairpin modified self-blocked crRNAs. Plot of Mean Raw Fluorescence Unit (RFU) for n=3 replicates at t=30 min is shown. NTC=No Target Control. FIG. 11C is a bar graph illustrating detection of SARS-CoV-2 RNA in 6 patient samples (3 pos, 3 neg as pre-determined by qPCR) using an embodiment of the CCR system of the present disclosure using a crGFP-14-3′ self-blocked hairpin modified crRNA.

FIGS. 12A and 12B illustrate detection of various concentrations of HIV target by hairpin modified blocked crRNAS, crGFP-14-3′, crGFP-28-3′ and crGFP-41-3′. FIG. 12A is a plot of mean fluorescence intensity with time for crGFP-14-3′, and FIG. 12B is a plot of RFU at t=60 min is indicated.

FIGS. 13A and 13B are bar graphs illustrating detection of a synthetic DNA resembling the N-gene of SARS-CoV-2 using CCR systems of the present disclosure at different temperatures. FIG. 13A illustrates the RFU of the single-point reading done after 60 min incubation. FIG. 13B is a plot of Signal:Noise ratio at each different temperature calculated by taking the ratio of the mean RFUs of 10 fM N gene target and the No Target Control (NTC). Higher signal:noise ratio indicates better detection.

FIG. 14 is a bar graph of detection of a synthetic HIV and SARS-CoV-2 N gene DNA at room temperature within 20 minutes using a CCR system of the present disclosure.

FIG. 15 is a graph comparing the detection sensitivity of embodiments of a CCR system of the present disclosure against a recently described detection system called “CONAN” for detection of a synthetic SARS-CoV-2 gene. Mean RFU (n=3) at time=30 min is plotted for each construct. Error bars represent SD. Data indicates that crGFP-14-3′ is able to distinguish between 1.5 pM CoV target and NTC much faster than crGFP-28-3′ or “CONAN.”

FIG. 16 illustrates embodiments of crRNA blocking moieties for embodiments of CCR systems of the present disclosure. Five different models (a-e) are illustrated, including a 24-mer phosphorothioate extension (a), a 31-mer DNA extension (b), a looped crRNA (c), a self-looping/blocking crRNA (d), and a biotinylated crRNA bound to streptavidin coated magnetic beads (e).

FIGS. 17A and 17B illustrate detection of various concentrations of HIV target by phosphorothioate modified blocked crRNAs. FIG. 17A is a plot of mean RFU with respect to time (n=3), and FIG. 17B is a heat map of the data in FIG. 17A.

FIGS. 18A and 18B illustrate detection of various concentrations of a synthetic SARS-CoV-2 target by crRNA modified by magnetic bead-biotin modified crRNA. FIG. 18A is a plot of mean RFU with respect to time (n=2), and FIG. 18B is a heat map of the data in FIG. 18A.

FIG. 19 illustrates 4 different models of blocked secondary DNA or RNA activators blocked with a linked hairpin blocking moiety. In the models illustrated single-stranded DNA (a) or RNA (b) activators can be blocked by adding a DNA or RNA hairpin-loop at their 3′-end (models (c) and (d)) or 5′-end (models (e) and (f)) and extending it such that a complementary RNA or DNA blocker blocks it through base-pairing. The non-complementary bulges placed within the RNA or DNA allow the blocked activator to become unblocked through the trans-cleavage activity of a primary CRISPR/Cas system. A blocked activator can be used in tandem with the blocked crRNA demonstrated earlier to have a significantly enhanced fluorescence signal for detection.

FIGS. 20A-D illustrate different embodiments of CCR systems (FIGS. 20A and 20D) and multiplexed orthogonal CRISPR-based target detection (CBTD) systems (FIGS. 20B and 20C) combining either a primary Cas12a system and secondary Cas13a system (FIG. 20C) or a primary Cas13a system and a secondary Cas12a system (FIG. 20B).

FIGS. 21A-21B illustrate detection of HIV RNA using a multiplexed orthogonal CBTD system employing a Cas13a based primary system and a Cas12a based secondary system.

FIG. 21A is a plot of mean RFU vs time at different concentrations (n=3), and FIG. 21B is a graph of mean RFU for 3 replicates at t=120 min. Error bars represent SD. NTC=No Target Control.

FIGS. 22A-22 B illustrate detection of the Tat gene in HIV-1 RNA using a multiplexed orthogonal CBTD system employing a Cas13a based primary system and a Cas12a based secondary system as in FIGS. 21A-21B. FIG. 22A is a graph of RFU vs time for 3 replicates, and FIG. 22B illustrates a diagram of a blocked secondary activator ssDNA.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genetics, biochemistry, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20-25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Prior to describing the various embodiments, the following definitions are provided and should be used unless otherwise indicated.

### Definitions

In describing and claiming the disclosed subject matter, the following terminology will be used in accordance with the definitions set forth below.

It must be noted that, as used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

The terms “comprise”, “comprising”, “including” “containing”, “characterized by”, and grammatical equivalents thereof are used in the inclusive, open sense, meaning that additional elements may be included. It is not intended to be construed as “consists of only.”

As used herein, “consisting of” and grammatical equivalent thereof exclude any element, step or ingredient not specified in the claim.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “optional” or “optionally” means that the subsequently described event or circumstance can or cannot occur, and that the description includes instances where said event or circumstance occurs and instances where it does not.

As used herein, “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), CRISPR RNA (crRNA), Trans-activating crRNA (tracrRNA), or coding mRNA (messenger RNA).

As used herein, the terms “guide polynucleotide,” “guide sequence,” or “guide RNA” can refer to any polynucleotide sequence having sufficient complementarity with a target polynucleotide sequence to hybridize with the target sequence and direct sequence-specific binding of a CRISPR/Cas complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide can include a nucleotide sequence that is complementary to a target DNA sequence. This portion of the guide sequence can be referred to as the complementary region of the guide RNA. In some contexts, the two are distinguished from one another by calling one the complementary region or target region and the rest of the polynucleotide the guide sequence or tracrRNA. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR (Nature 517, 583-588 (29 Jan. 2015) or suppression (Cell Volume 154, Issue 2, 18 Jul. 2013, Pages 442-451). A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR/Cas complex to a target sequence may be assessed by any suitable assay. For example, the components of a CRISPR system sufficient to form a CRISPR/Cas complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR/Cas complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioate, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “specific binding” or “preferential binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” or “an aspect” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

### Discussion

In accordance with the purpose(s) of the present disclosure, as embodied and broadly described herein, embodiments of the present disclosure, in some aspects, relate to CRISPR/Cas chain reaction (CCR) systems and methods employing a secondary crRNA/blocker/activator system to create a CRISPR/Cas chain reaction to amplify a signal produced by a primary CRISPR-based target detection (CBTD) system for detection of a target (e.g., a target polynucleotide). The CCR systems of the present disclosure substantially amplify the detection sensitivity compared to the CRISPR-based target detection (CBTD) system alone (e.g., by producing a detectable signal in shorter amount of time, producing detectable signal with lower starting amount of target, producing a stronger detectable signal, and the like, and combinations of these). The systems and methods of the present disclosure can also be optimized to increase both sensitivity and specificity of detection.

The systems of the present disclosure provide CRISPR/Cas chain reaction (CCR) systems for amplifying the detection sensitivity of a primary CRISPR-based target detection (CBTD) system to detect targets, such as target polynucleotides, with high sensitivity and specificity. These CCR systems are universal and can be quickly adapted for use with any primary CRISPR based detection system, including commercially available CBTD systems, other CBTD systems in development, and the like. They can also be combined with CBTD systems designed to detect an array of targets, from polynucleotide targets like single or double-stranded DNA, RNA, and DNA/RNA heteroduplexes, as well as CBTD systems designed to detect proteins and other molecules. The CCR systems increase the sensitivity to reduce or eliminate the need for any target pre-amplification step such as PCR, RT-LAMP, and the like. Moreover, the embodiments disclosed herein can be prepared for convenient distribution and point-of-care (POC) applications. Such embodiments are useful in multiple scenarios in human health including, for example, viral detection, bacterial strain typing, sensitive genotyping, and detection of disease-associated cell free DNA.

Overview

The breakthrough of CRISPR/Cas (clustered regularly interspaced short palindromic repeats/CRISPR-associated) systems has transformed the slow-progressing field of genome engineering with astronomical applications in biology, agriculture, biotechnology, diagnostics, and treatment of genetic disorders¹⁻⁵. Originally derived from different species of bacterial adaptive immune systems, the CRISPR/Cas technology works by introducing a Cas nuclease that acts like molecular scissors and a short crRNA that serves as a guide by binding with Cas and directing the crRNA/Cas complex to the target site. This complex then creates double-stranded cuts in the DNA or a single-stranded cut in the RNA. This specific target recognition and cleavage is also referred to as ‘cis-cleavage’. Most type V and type VI CRISPR/Cas systems exhibits an additional non-specific collateral cleavage activity of single-stranded nucleic acids immediately after the specific target recognition, referred as ‘trans-cleavage’ activity. The CRISPR/Cas systems are recently being explored as novel POC in vitro diagnostics and have fewer regulatory challenges compared to gene editing.

Type V and VI CRISPR/Cas systems have recently emerged as diagnostics for detecting DNA and RNA, respectively. That is because most type V and VI CRISPR/Cas complexes, when specifically bound with their specific target nucleic acid sequence, activate a secondary collateral nuclease activity that can rapidly cleave single-stranded nucleic acids in a multiple turnover manner. By designing single-stranded nucleic acid-based reporters, the trans-cleavage activity can be monitored with fluorescence-based and lateral flow-based assays (and other reporter systems) indicative of the presence or absence of a target. Most variants of type V CRISPR systems, including Cas12a-k and Cas14a-h, recognize dsDNA and possess trans-cleavage of ssDNA. Similarly, CRISPR/Cas13a-d, clustered as type VI CRISPR systems, display ssRNA trans-cleavage activity after binding to the target ssRNA sequence.

The Cas12a- and Cas13a-based detection platforms, coupled with reverse transcriptase and isothermal DNA amplification strategies, have recently received emergency use authorization by the FDA for detecting SARS-CoV-2 genomic RNA, highlighting the urgent need for improved, rapid, point-of-care diagnostic systems. Such systems, while especially important for management and mitigation of a global pandemic, are also useful for diagnosis of other diseases and conditions. However, traditional CRISPR/Cas detection systems typically have relatively low sensitivity of detection in the picomolar to the nanomolar range, and therefore, they are often coupled with a combination of target pre-amplification techniques with and/or CRISPR/Cas modifications to improve sensitivity, necessitating the use of multiple laboratory equipment to successfully conduct nucleic acid tests for COVID-19. The development of a highly sensitive, specific, point-of-care CRISPR-based system for early detection of targets, such as target polynucleotides like SARS-CoV-2 RNA, that does not require costly/time-consuming pre-amplification techniques has the potential to transform the field of diagnostics.

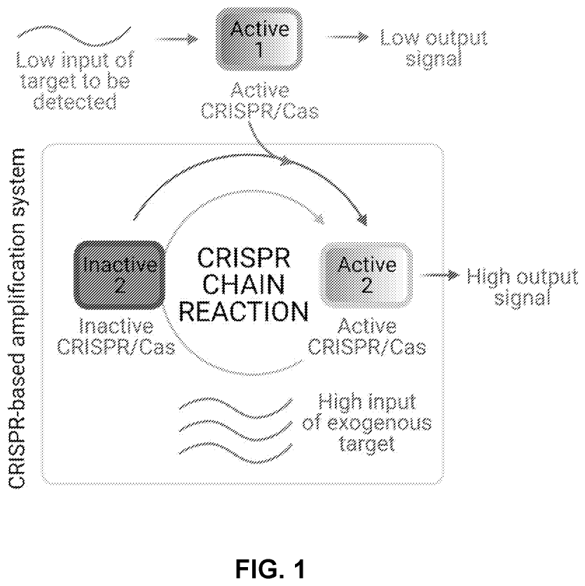

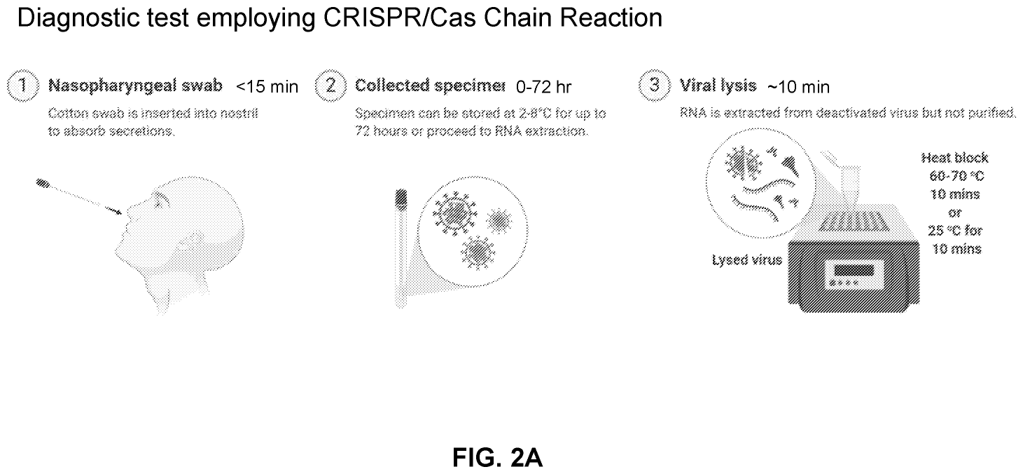

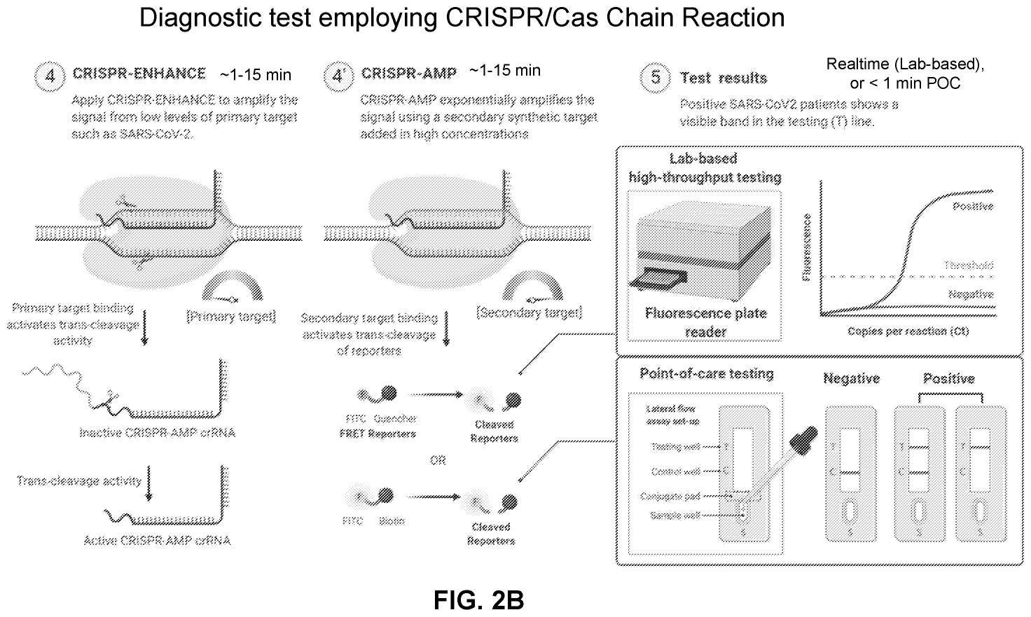

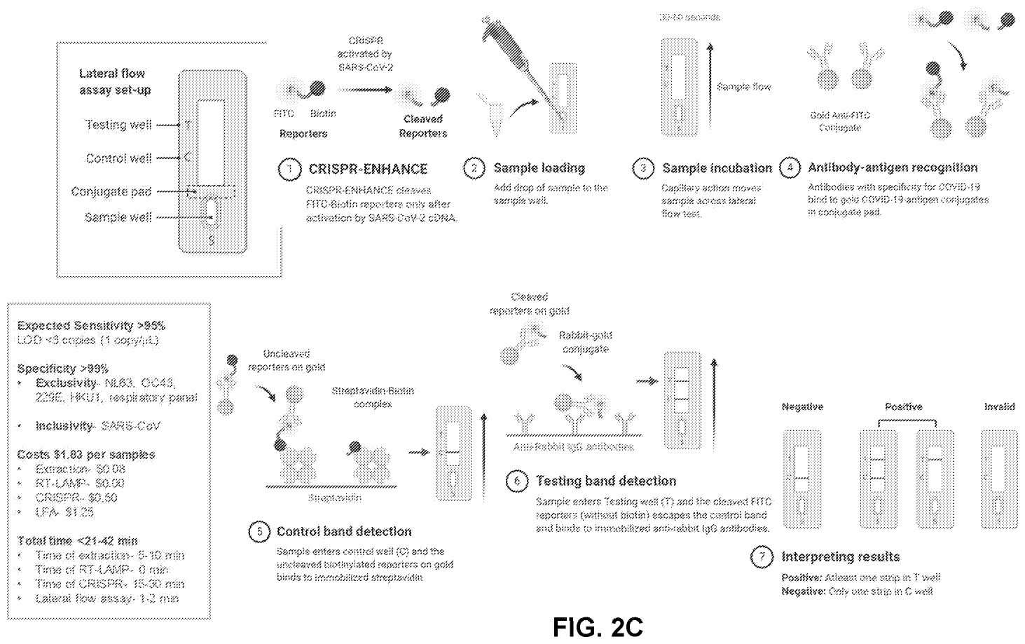

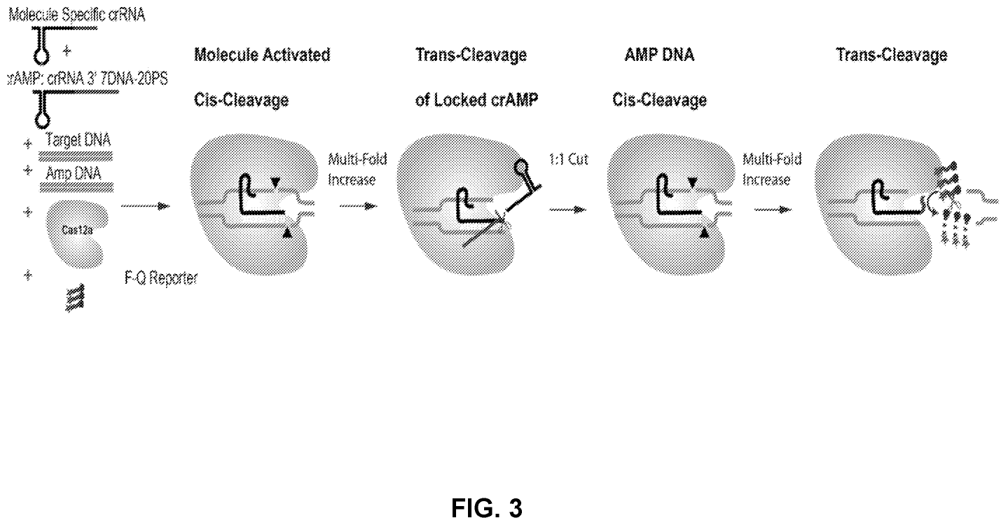

The present disclosure provides a CRISPR-based diagnostic method called CRISPR/Cas Chain Reaction (CCR) that can be combined with a primary CRISPR-based detection system (CBTD) system and that can also be amplification-free. Briefly described, and as illustrated in FIG. 1, FIGS. 2A-2B, and FIG. 3, embodiments of the CCR system and method combine a primary, on-target CRISPR/Cas system designed to detect a specific target with a ‘locked’ secondary CRISPR/Cas system that includes a crRNA that is locked for activity. The locked, or inactive, secondary system can include exogenously added secondary DNA activators and the secondary crRNA that is “locked” or “blocked” for activity. The locked secondary system becomes unlocked and produces an enhanced signal only after the primary CBTD system detects its target and initiates non-specific collateral (or “trans”) cleavage with an activated primary CRISPR/Cas complex. As shown in FIG. 1, the activated primary CRISPR/Cas complex activates the inactive secondary CRISPR/Cas complex which then initiates a chain reaction in which additional inactive secondary complexes can be activated both by activated primary complexes and activated secondary complexes. All of the activated primary and secondary complexes can then activate a detection signal which produces a high-output signal. A general description of the use of the CCR system and method of the present disclosure to detect a target in a patent sample is provided in FIGS. 2A-2C, where FIG. 2A illustrates sample collection and processing (steps 1-3). FIG. 2B illustrates the combined primary CBTD system (step 4) and the CCR system (“CRISPR-AMP”, step 4′), and various methods/systems for analyzing the signal produced by the system (step 5). FIG. 2C illustrates combination of the methods and systems of the present disclosure with a lateral flow assay for target detection.

FIG. 3 provides another illustration of an embodiment of a CCR system of the present disclosure combined with a primary CBTD system to detect a target with amplified signal. Panel (a) shows the different elements of a combined CBTD system and CCR system, panel (b) illustrates the primary CBTD target detection and cis-cleavage, panel (c) illustrates trans cleavage of the locked secondary crRNA of the CRR system, releasing it to bind with Cas and activator in panel (d) to form an activate secondary CCR system in panel (e), which is then able to cleave multiple probes. Not shown is that the activated secondary CCR system in panel (e) can also cleave additional locked secondary crRNA's producing additional activated secondary CRR systems, and both activated primary CBTD systems and CCR systems can cleave probe, allowing for multi-stage amplification.

As described in the Examples below, in embodiments, the CCR platform was able to perform amplification-free detection of attomolar levels of a wide variety of synthetic DNA and RNA targets including Malaria, HIV-1, and SARS-CoV-2 within 60-90 minutes at room temperature. The CCR platform described in the present disclosure represents a universal CRISPR/Cas amplification system and can be rapidly coupled with any CRISPR-based target detection system to enhance its sensitivity of detection.

CRISPR/Cas Chain Reaction (CCR) Systems

The general components of embodiments of CCR systems of the present disclosure will be described here, and additional details about the different components will also be provided below.

A CCR system of the present disclosure for amplifying the detection sensitivity of a primary CBTD is designed to work with a CBTD system that includes the following elements: a plurality of CRISPR-associated (Cas) enzymes with activatable trans-cleavage activity (in embodiments, the Cas enzymes can be provided with the primary CBTD system, the CCR system, or both) and a plurality of primary CRISPR RNAs (crRNA) configured to detect a specified target. The primary crRNAs are capable of forming a complex with one of the Cas enzymes to form a primary crRNA/Cas complex, and each primary crRNA has a primary guide sequence configured to bind the target. In such systems binding of the primary crRNA/Cas complex to the target activates the trans-cleavage activity of the Cas enzyme to produce an activated primary crRNA/Cas complex. In traditional CBTD systems, the activated primary crRNA/Cas complex would then be able to cleave probes with a portion configured to be cleavable by the Cas trans cleavage. In embodiments, probes can be provided with the primary CBTD system, the CCR system, or both. In the CCR systems of the present disclosure, while the activated primary crRNA/Cas complex can still cleave probes to produce a detectable signal, the activated primary crRNA/Cas complex can also activate the CCR system, which then creates the CRISPR-mediated chain reaction to amplify the signal multi-fold.

The CCR system of the present disclosure also includes a plurality of crRNAs, and these secondary crRNAs are different from the primary crRNAs. The secondary crRNAs have a guide sequence configured to bind an activator sequence instead of the target sequence. The secondary crRNAs are also capable of forming a complex with one of the Cas enzymes to form a secondary crRNA/Cas complex, but are prevented from becoming activated by a blocking mechanism. The CCR system also includes a plurality of the activators, each activator having an oligonucleotide element complementary to and configured to bind the secondary crRNA, so that when binding of the secondary crRNA to the Cas and to the activator is possible, this activates the trans-cleavage activity of the Cas enzyme to produce an activated secondary crRNA/Cas complex. The CCR system further includes a plurality of blocking moieties bound either to the secondary crRNA or to the activator such that the bound blocking moiety prevents binding of the secondary crRNA to the Cas enzyme or prevents binding of the secondary crRNA/Cas complex to the activator. Each of these blocking moieties has a cleavable sequence configured to be cleaved by an activated Cas enzyme of an activated primary crRNA/Cas complex or an activated secondary crRNA/Cas complex. Cleavage of the cleavable sequence of the blocking moiety releases the blocking moiety allowing the secondary crRNA to bind another of the Cas enzymes and an activator to produce an activated secondary crRNA/Cas complex, resulting in the CRISPR/Cas chain reaction that produces additional activated secondary crRNA/Cas complexes. All of these additional activated secondary crRNA/Cas complexes can activate additional secondary crRNA/Cas complexes and can cleave/activate probes. As mentioned above, the CCR system also include the probes (which can either be included in the primary CBTD system, the CCR system, or both). The probes have an oligonucleotide element labeled with a detectable label, and the probe is configured to be cleaved by any of the activated Cas enzymes of the activated primary crRNA/Cas complexes or the activated secondary crRNA/Cas complexes. When cleaved, the probe is able to generate a detectable signal or a detectable molecule. Due to the cascade of secondary crRNA/Cas activation, the CCR system allows exponential cleavage of probes thereby amplifying the detection sensitivity compared to the CRISPR-based target detection (CBTD) system alone.

Further description and examples of the various components of the CCR systems of the present disclosure are provided below.

Primary CRISPR-Based Target Detection (CBTD) System

The CCR systems and methods of the present disclosure are designed to be used in conjunction with a primary CBTD system that is designed to use a crRNA/Cas complex system to detect a specified target. Such systems are known in the literature and described herein. The primary CBTD system can include commercially available systems such as some of the systems described above as well as a system previously developed with enhanced sensitivity and referred to as CRISPR-ENHANCE described in PCT application PCT/US2020/059577 (publication WO 2021/092519), hereby incorporated by reference herein.

Briefly described, a primary CBTD system that can be used with the CCR systems or the multiplexed CBTD systems described below, includes the following elements: a primary crRNA, a Cas enzyme, and probes. These various elements will be described in greater detail below with respect to primary CBTD systems, CCR systems, kits, and methods of the present disclosure, as well as multiplexed CBTD systems of the present disclosure.

As shown in FIG. 2, the primary CBTD system is illustrated in

Probe

As used herein, a “probe” refers to a polynucleotide-based molecule that can be cleaved by an activated CRISPR-associated (Cas) enzyme with a trans-cleavage activity to produce a detectable signal or a detectable molecule. A detectable signal may be any signal that can be detected using optical, fluorescent, chemiluminescent, electrochemical or other detection methods known in the art. The probe comprises an oligonucleotide element. In one embodiment, a first end of the oligonucleotide element in the probe is linked to a fluorophore; and a second end of the oligonucleotide element in the probe is linked to a quencher of the fluorophore. In one embodiment, the probe further comprises biotin. In some embodiments of the present disclosure, the probes are configured to be cleaved by a Cas enzyme in either a primary crRNA/Cas complex or a secondary crRNA/Cas complex, such that the signal can be produced upon primary binding of target as well as upon binding of secondary crRNA/Cas complex to activator to produce amplified signal.

Quenching of the fluorophore can occur as a result of the formation of a non-fluorescent complex between the fluorophore and another fluorophore or nonfluorescent molecule. This mechanism is known as ground state complex formation, static quenching, or contact quenching. Accordingly, the oligonucleotide element may be designed so that the fluorophore and quencher are in sufficient proximity for contact quenching to occur. Fluorophores and their cognate quenchers are known in the art and can be selected for this purpose by one having ordinary skill in the art. Upon activation of the CRISPR-associated enzyme disclosed herein, the oligonucleotide-based probe is cleaved thereby severing the proximity between the fluorophore and quencher needed to maintain the contact quenching effect. Accordingly, detection of the fluorophore may be used to determine the presence of a target molecule in a sample. In one embodiment, the fluorophore is selected from the group consisting of FITC, HEX and FAM, and the quencher is selected from the group consisting of BHQ1, BHQ2, MGBNFQ, and 3IABkFQ. In one embodiment, a first end of the oligonucleotide element in the probe is linked to a fluorophore; a second end of the oligonucleotide element in the probe is linked to a quencher; and the probe further comprises biotin. In one embodiment, a fluorophore-quencher probe is within the crRNA and the quencher was only cleaved in the presence of a target polynucleotide.

A detectable molecule may be any molecule that can be detected by methods known in the art. In one embodiment, the detectable molecule is one member of a binding pair and can be detected by binding to another member of the binding pair. Examples of binding pairs include, but are not limited to, antibody-antigen pairs, enzyme-substrate pairs, receptor-ligand pairs, and streptavidin-biotin.

In one embodiment, the detectable label is a label selected from the group consisting of FAM-biotin, FITC-biotin, FAM-biotin-quencher, and FITC-biotin-quencher. In one embodiment, a first end of the oligonucleotide element in the probe is linked to FITC, and a second end of the oligonucleotide in the probe is linked to biotin.

In one embodiment, the oligonucleotide element in the probe is ssDNA or RNA, depending on if the Cas enzyme's trans cleavage activity preferentially cuts DNA or RNA. If the Cas enzyme preferentially cleaves DNA (e.g., Cas12 enzymes), the oligonucleotide element of the probe can be ssDNA, preferably with an A/T rich sequence. If the Cas enzyme preferentially cleaves RNA (e.g., Cas13 enzymes), the oligonucleotide element of the probe can be RNA (preferably with A/U rich sequences). In one embodiment, if the Cas enzyme is Cas12 (e.g., a Cas12a enzyme) the ssDNA in the probe includes at least 55%, 60%, 65%, 70%, 75%, 80%, 90%, or 95% of A and/or T. In one embodiment, the ssDNA consists of A and/or T. In one embodiment, the oligonucleotide element is TTATT. In one embodiment, if the Cas enzyme is Cas13 (e.g., a Cas13a enzyme) the ssDNA in the probe includes at least 55%, 60%, 65%, 70%, 75%, 80%, 90%, or 95% of A and/or U. In one embodiment, the ssDNA consists of A and/or U.

In one embodiment, the probe comprises FAM-TTATT-3IABkFQ. In another embodiment, the probe comprises FITC-TTATT-Biotin. In another embodiment, the probe comprises FAM-TTATTA(internal biotin)T-3IABkFQ.

CRISPR-Associated Enzyme

CRISPR-associated (Cas) enzymes (also known as CRISPR effector protein) are enzymes which can bind to a crRNA containing a guide sequence to form a crRNA/Cas or (CRISPR/Cas) complex, which can bind to target polynucleotide sequence that is complementary to the guide sequence of the crRNA. The Cas enzymes useful for the systems and methods of the present disclosure possess both cis- and trans-cleavage activity. In such embodiments, an activated CRISPR-associated enzyme remains active following binding of a target sequence and continues to non-specifically cleave non-target oligonucleotides. This guide molecule-programmed trans-cleavage activity provides an ability to use CRISPR/Cas systems to detect the presence of a specific target oligonucleotide to trigger non-specific polynucleotide cleavage that can serve as a readout.

In one embodiment, the Cas is a class V or VI Cas enzyme having trans cleavage activity. In embodiments, the Cas enzyme is a Cas 12, Cas 13, or Cas14 enzyme. In some embodiments, the target is a ssDNA, dsDNA, DNA/RNA heteroduplex sequence and the Cas12 enzyme is selected from Cas 12a, Cas12b, Cas12c, Cas12d, Cas12e, Cas12f, Cas12g, Cas12h, Cas12i, Cas12j (Cas12j1-Cas12j10), or Cas12k. In another embodiment, the Cas enzyme is Cas 12a. In some embodiments, the target is ssRNA and the Cas12 enzyme is Cas12g. in some embodiments, the target is a ssDNA, dsDNA, DNA/RNA heteroduplex sequence and the Cas14 enzyme is selected from Cas 14a (Cas14a1-Casa6), Cas14b (Cas14b1-Casb16), or Cas14c (Cas14c1-Cas14c2). In yet other embodiments, the target is an RNA sequence and the Cas enzyme is a Cas13 enzyme selected from the group of Cas13 enzymes selected from: Cas13a, Cas13b, Cas13c, or Cas13d.

In most of the embodiments of the CCR system it is contemplated that the Cas enzyme is the same for both the primary CBTD system and the CCR system. Thus, if the primary CBTD system is designed to be used with a Cas12a enzyme, then the CCR system will be designed to also be used with the same Cas12a enzyme. Thus, the same Cas enzyme that is added with the CBTD system will also be utilized by the CCR system. In some embodiments, excess of the Cas enzyme is added with the CCR system. This design is useful because any of the activated crRNA/Cas complexes that have bound a target/activator (and thus activated the Cas trans cleavage activity) can cleave probes to produce detectable signal and can cleave additional blocker moieties, thereby freeing more of the secondary crRNA to bind with Cas and activator in a chain reaction.

However, in some embodiments, such as the multiplexed CBTD systems described below that combine a primary and secondary CBTD systems with different Cas enzymes. In such embodiments, it is contemplated that the secondary system may include a different Cas enzyme. In other words, the secondary system is activated by the Cas enzyme of the primary system, but the secondary system includes a secondary Cas enzyme (different from the first enzyme) that cleaves the probe/reporter. These systems don't have the same cascade effect since the activated secondary crRNA/Cas systems cannot create a domino effect of activating additional secondary crRNA/Cas systems. In such embodiments, the first crRNA/Cas system can only be activated by target detection, the second crRNA/Cas system can only be activated by an activated primary crRNA/Cas system, and the detection probes can only be activated by the activated secondary crRNA/Cas systems. However, as described in greater detail below, these systems are useful in situations in which it is desired to detect a target RNA molecule without using reverse transcriptase. In such embodiments, a primary crRNA/Cas system can be used that specifically targets and cleaves RNA, such as Cas13 complexes. However, the primary Cas may be less active (e.g., have lower trans-cleavage activity) than other forms of Cas, such as Cas12. In that case, the secondary CBTD system can employ a more active secondary Cas, such as Cas 12, so that once the secondary CBTD system is activated, the secondary Cas enzyme with cleave the probe with high activity for improved detection.

crRNA

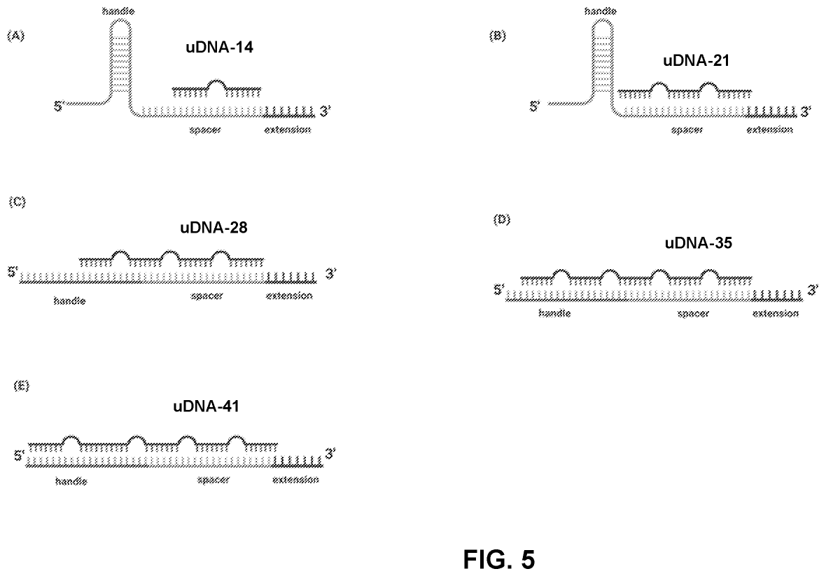

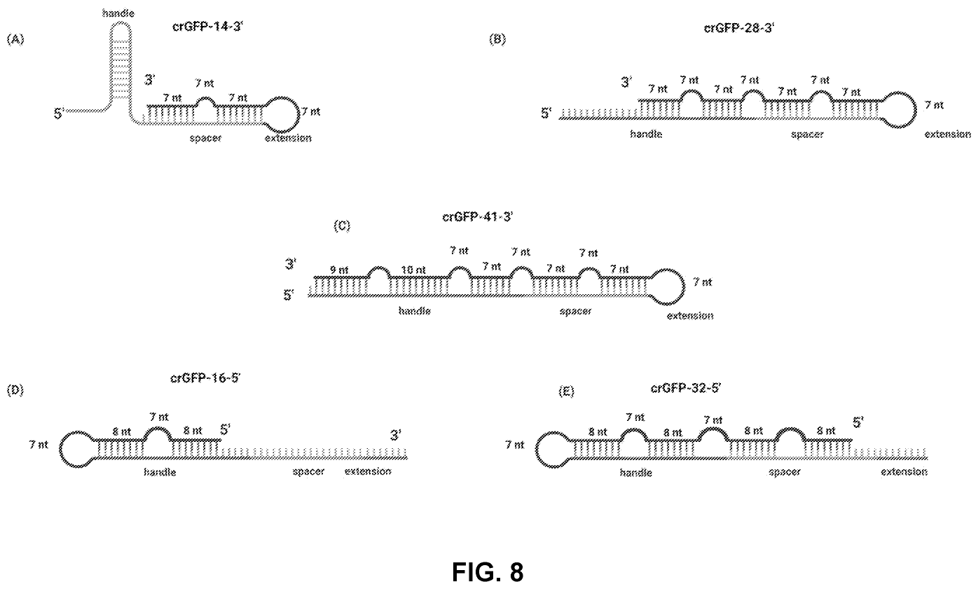

The present disclosure provides CRISPR RNAs (crRNA) including a guide (or sometimes referred to as “spacer” as in FIGS. 5A-5E and FIGS. 8A-8E) sequence and a conserved sequence, wherein the guide sequence is configured to bind to a target polynucleotide or an activator, and the conserved sequence (usually forming the handle portion) is conserved among crRNA from closely related bacterial species. In the present disclosure the crRNA can be a primary crRNA of a primary CBTD system and/or a secondary crRNA of a CCR system.

In some embodiments, the degree of complementarity between a guide sequence and its corresponding target sequence/activator sequence, when optimally aligned using a suitable alignment algorithm, is about or more than about 50%, 60%, 75%, 80%, 85%, 90%, 95%, 97.5%, 99%, or more. In one embodiment, the degree of complementarity between a guide sequence and its corresponding target sequence is about or more than about 50%, 60%, 75%, 80%, 85%, 90%, 95%, 97.5%, 99%, or more.

In some embodiments, a crRNA is about 42 base pairs. In embodiments, the length of the a guide sequence of the crRNA is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the guide sequence is less than about 75, 50, 45, 40, 35, 30, 25, 20, 15, 12, or fewer nucleotides in length. In one embodiment, the guide sequence is 10-30 nucleotides long. In some embodiments, the guide sequence includes an extension sequence connected to the 3′ end of the guide sequence which serves to further increase trans cleavage activity of the Cas enzyme.

In some embodiments, the crRNA can be modified have an extension sequence on the 3′ end of the guide sequence. This has been found to enhance target binding and activation of trans cleavage activity of the modified crRNA. Embodiments of such modified crRNA are described in PCT application PCT/US2020/059577 (publication WO 2021/092519), incorporated by reference above.

Activators

As used herein activators are a component of a CCR system designed to activate a secondary crRNA/Cas complex. In embodiments, the activators are polynucleotides having a sequence complementary to a secondary crRNA, such that the secondary crRNA can bind the activator, thereby activating the trans-cleavage activity of a Cas enzyme in the secondary crRNA/Cas complex. More details about the activators are described in the example and figures.

Blocking Moieties

As used herein blocking moieties are a component of a CCR system designed to prevent activation of the secondary crRNA/Cas complex by preventing either binding of the secondary crRNA to the Cas enzyme or binding of the secondary crRNA to the activator. In embodiments, the blocking moiety can be bound to the crRNA guide sequence to block it from binding the activator or the Cas enzyme. In embodiments, the blocker can be a polynucleotide with complementary portions to the crRNA guide sequence such as shown in FIGS. 5 and 8. In other embodiments, the blocker can be bound to the activator to prevent crRNA binding. In yet other embodiments, the blocker can be bound to the crRNA to block it from complexing with the Cas enzyme.

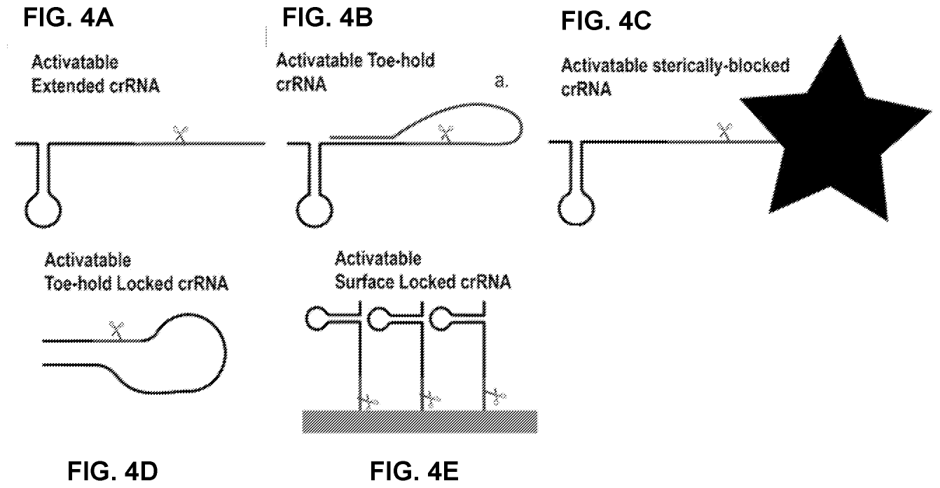

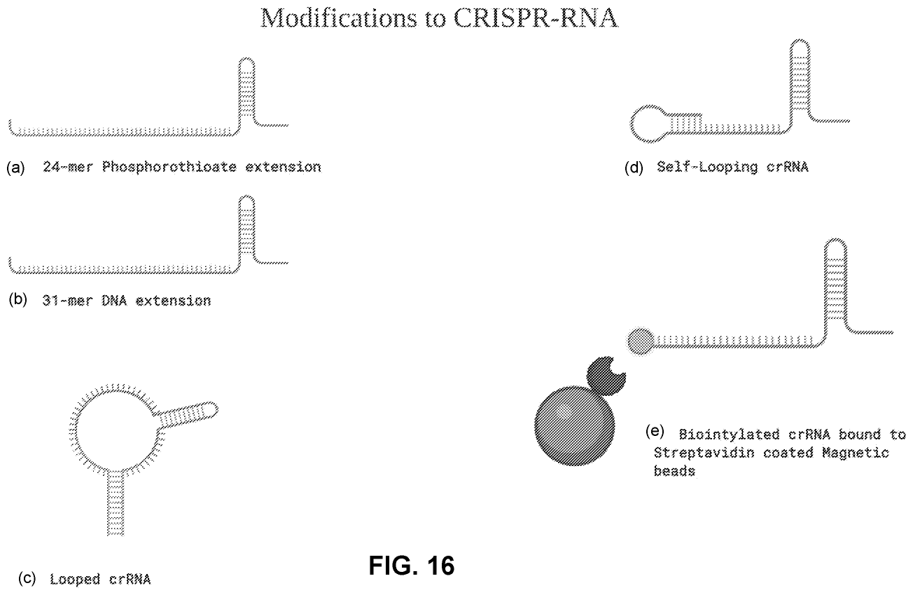

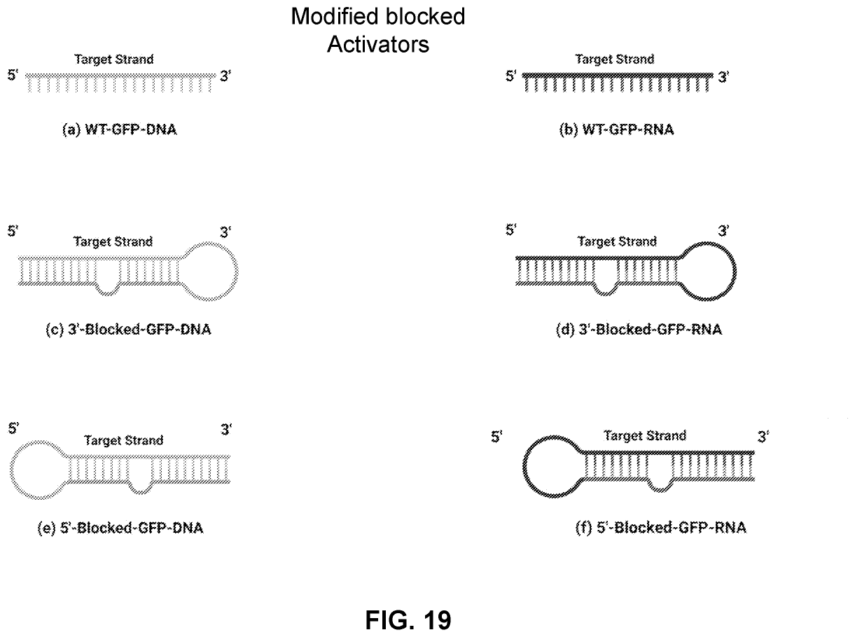

Some embodiments of different blocking moieties are illustrated generally in FIGS. 4A-4E and also FIG. 19. Some examples of blocking moieties include, but are not limited to, a phosphorothiolated crRNA having a phosphorothiolated extension sequence attached to the crRNA by a cleavable linker (FIG. 4A and FIG. 16, model (a)) that in some embodiments may have a partially complementary region at an opposite end that is able to loop around and bind to the crRNA (FIG. 4B); a large molecule that blocks by steric hinderance attached to an end of the crRNA with a cleavable linker (FIG. 4C and FIG. 16, model (e)); a complementary nucleotide sequence that forms a hairpin loop with the crRNA and is attached with a cleavable linker (FIG. 4D and FIG. 16, model (d)); and a surface (e.g., a substrate surface), where the crRNA is connected to the surface by a cleavable linker, such that the crRNA is prevented from interacting with activator and/or Cas enzymes due to being tethered to the surface (FIG. 4E). Other blocking moieties, such as those shown in FIGS. 16, models (b) and (c) can be contemplated by a skilled artisan and are within the scope of this disclosure. Some examples of blocking moieties configured to block the activator rather than the crRNA are illustrated in FIG. 19.

So that the blocking moiety can be released to allow activation of the secondary crRNA/Cas complex, the blocking moiety is configured to be cleaved by an activated Cas enzyme (e.g., an activated Cas enzyme in an activated primary crRNA/Cas complex and/or in an activated secondary crRNA/Cas complex). In embodiments the blocking moiety has a cleavable sequence configured to be cleaved by an activated Cas of an activated primary crRNA/Cas complex or an activated secondary crRNA/Cas complex. In this way, cleavage of the cleavable sequence of the blocking moiety by the activated Cas enzyme releases the blocking moiety allowing the secondary crRNA to bind another of the Cas enzymes and activator to form another secondary crRNA/Cas complex that can in turn cleave more blocking moieties, resulting in a CRISPR/Cas chain reaction that produces additional activated crRNA/Cas complexes. The cleaving of blockers that results binding of crRNA/Cas complexes to activators also allows these activated crRNA/Cas complexes with activated trans cleavage activity to cleave the probes resulting in enhanced signal.

In some embodiments, such as shown in FIGS. 5 and 8, the blocking moiety is a blocking nucleotide sequence bound to the secondary crRNA that prevents binding of the secondary crRNA to the activator. In embodiments, the blocking nucleotide sequence includes one or more complementary segments bound to the secondary crRNA and one or more non-complementary, unbound segment having a sequence cleavable by an activated Cas enzyme. Cleavage of the one or more non-complementary segments of the blocking nucleotide by the activated Cas enzyme releases the secondary crRNA from the blocking nucleotide such that the secondary crRNA can bind the activator. In embodiments the one or more complementary segments are each about 3-41 nucleotides long, such as, but not limited to about 3-20, about 4-10, about 7 or 8, and the like (for embodiments where the CCR system is designed to be used in an assay conducted at elevated temperatures, such as 38-65 C, or where the blocking sequence has a lower Tm, then longer complementary segments, such as up to about 41 nucleotides long might be used). In embodiment, the one or more non-complementary, unbound segments are each from 2-40 nucleotides long, such as, but not limited to about 2-30, about 3-12, about 7, and the like. According to certain embodiments, the blocking nucleotide sequence has about 2-5 complementary segments, each about 7-10 nucleotides long (e.g., consecutive complementary nucleotides), and has about 1-5 non-complementary, unbound segments, each about 7-10 nucleotides long. In embodiments, the complementary and non-complementary segments are alternating (e.g., one non-complementary segment in between two complementary segments). Other configurations are within the scope of the present disclosure.

FIG. 5 illustrates 5 different models of blockers that have complementary DNA sequences of various lengths that contain non-complementary bulges within them and are unlinked to the end of the crRNA, termed uDNA blockers for “unlinked blockers”. The uDNA blocker moieties block the crRNA activity through base pairing and inactivate it. The blocked crRNA can be activated again by the trans-cleavage activity of a primary CRISPR/Cas system that cleaves the ssDNA or ssRNA bulges within the uDNA blocker. The unblocked secondary crRNA is then able to recognize and cleave an excess of secondary dsDNA-activator that is exogenously added to the system. This starts a chain reaction of CRISPR/Cas activity in which more secondary crRNAs are unblocked and an excess of fluorescent reporters are cleaved thereby producing a high output signal that can be detected using a fluorescence reader.

Similarly, FIG. 8 illustrates 5 different models of blockers that, like the uDNA/RNA sequences of FIG. 5, have complementary DNA sequences of various lengths that contain non-complementary bulges within them, but differ from the uDNA/RNA blockers in that they are linked to the 5′ or 3′ end of the crRNA forming a hairpin loop formation, and sometimes referred to herein as modified hairpin blocked crRNAs or self-blocked crRNAs. In embodiments of such modified hairpin blocked crRNAs the hairpin loops can be further extended to have different lengths of a complementary DNA sequence with bulges placed at regular intervals. These DNA extensions block the crRNA through base pairing and inactivate it. The blocked secondary CRISPR RNA can be activated again by a primary CRISPR/Cas system that cleaves the linker and/or ssDNA bulges in the extension through trans-cleavage activity. The unblocked CRISPR RNA is then able to recognize and cleave an excess of secondary dsDNA activator present in the system. This starts a chain reaction of CRISPR/Cas activity in which more secondary crRNAs are unblocked and an excess of fluorescent reporters are cleaved thereby producing a much higher output signal.

Kits

Embodiments of the present disclosure also include kits including a CCR system of the present disclosure and a primary CBTD system. In embodiments, the kits also include instructions for using the primary CBTD system and the CCR system to detect a target in a sample. In embodiments, the primary CBTD system includes a plurality of Cas enzymes with activatable trans cleavage activity and a plurality of crRNAs capable of forming a complex with one of the Cas enzymes to form a primary crRNA/Cas complex having a primary guide sequence configured to bind a target, such that binding of the primary crRNA/Cas complex to the target activates the trans-cleavage activity of the Cas enzyme to produce an activated primary crRNA/Cas complex. In some embodiments, the kit comprises: an excess of the Cas enzyme, an excess of the primary crRNA, and an excess of activators. For purposes of the present disclosure “excess” indicates there are more of a particular element than needed for reaction with a corresponding element.

In embodiments the primary CBTD system is a commercially available CBTD system. In some embodiments, the primary crRNA is a polynucleotide extension sequence linked to a 3′-end of the guide sequence, and the polynucleotide extension sequence has a ssDNA or ssRNA having 1-31 nucleotides. In some embodiments, if the Cas enzyme is a DNA-targeting nuclease, the polynucleotide extension sequence comprises a ssDNA having a sequence of at least 80% A and/or T, and if the Cas enzyme is an RNA-targeting nuclease, the polynucleotide extension sequence comprises a ssRNA having a sequence of at least 80% A and/or U.

Methods

Methods of the present disclosure include using the CRISPR/Cas chain reaction (CCR) systems of the present disclosure to amplify the detection sensitivity of a primary CRISPR-based target detection (CBTD) system. Methods of amplifying the detection sensitivity of a primary CBT system include combining a primary CBTD system with a sample including a target to be detected and a CCR system of the present disclosure. It is contemplated that the signal generated by the CCR system will be greater than a signal produced from the primary CBTD system alone in the same amount of time.

In embodiments, a detectable, diagnostically relevant signal will be produced in a shorter amount of time and/or the signal will be more diagnostically relevant than a signal from the primary CBTD system. In embodiments, the signal to be detected can be, but is not limited to: fluorescence, luminescence, a chemical signal, a magnetic signal, a colorimetric signal, other optical signals, pH changes, temperature changes, electrochemical signals, and combinations of these

In embodiments, the method can be performed without any pre-amplification of the target prior to combination with the primary CBTD system and the CCR system. According to some embodiments, the method can be performed in a one-pot-reaction with the components of the CBTD system and the CCR system combined together sequentially or simultaneously. In embodiments, the detection methods with the CCR system of the present disclosure are performed at a temperature of about 20° C. to 75° C., such as from about 20° C. to 50° C., about 25° C. to 40° C., and the like. In embodiments, it can be performed at about room temperature (e.g., about 20-25° C.). In embodiments, the signal detected is selected from, but not limited to, fluorescence, luminescence, a chemical signal, a magnetic signal.

Multiplexed CRISPR-Based Target Detection (CBTD) Systems and Methods

Embodiments of the present disclosure also include dual/tandem CRISPR-based target detection systems (CBTD) that combine a primary CBTD system and secondary CBTD system. These systems are similar to the CCR systems except they do not typically involve a chain reaction of the activation of the secondary CBTD system, as in the CCR systems described herein (though they can be combined with a CCR system for a 3-component system). Instead these multiplexed CBTD systems combine two different/orthologous crRNA/Cas systems to provide certain advantages from each type of system. This multiplexed approach allows combining features/advantages of one Cas system, such as a Cas13 or Cas14 system with that of a Cas12. For instance, Cas 13a can detect RNA targets, while Cas 12a preferentially cuts dsDNA, ssDNA, or RNA/DNA heteroduplex. Thus, if the primary target is a RNA, DNA, etc., a Cas enzyme can be selected form the primary CBTD that is appropriate for that target. The secondary CBTD system can be designed to be activated by the primary CBTD system (e.g., a blocking moiety cleaved by the primary crRNA/Cas complex), while the probes can be designed to be cleaved by the secondary crRNA/Cas complex. This may be beneficial, for instance, with an RNA target to avoid the need for a reverse transcriptase step, such as described above.

Briefly described, the multiplexed CBTD systems can include a primary CBTD system and secondary CBTD system. The primary CBTD system includes a plurality of primary Cas enzymes with activatable trans-cleavage activity and a plurality of primary crRNAs capable of forming a complex with one of the primary Cas enzymes to form a primary crRNA/Cas complex. Each primary crRNA can have a primary guide sequence configured to bind a target, such that binding of the primary crRNA/Cas complex to the target activates the trans-cleavage activity of the primary Cas enzyme to produce an activated primary crRNA/Cas complex.

The secondary CBTD system includes a plurality of secondary Cas enzymes with activatable trans cleavage activity and that are different from the primary Cas enzyme. The secondary CBTD system also includes a plurality of secondary crRNAs capable of forming a complex with a secondary Cas enzyme to form a secondary crRNA/Cas complex. The secondary crRNAs each have a secondary guide sequence configured to bind an activator. The secondary CBTD system also includes a plurality of activators, each activator having an oligonucleotide element complementary to and configured to bind the secondary crRNA, such that binding of the secondary crRNA/Cas complex to the activator activates the trans-cleavage activity of the secondary Cas enzyme to produce an activated secondary crRNA/Cas complex. The system further includes a plurality of blocking moieties bound either to the secondary crRNA or to the activator such that the bound blocking moiety prevents binding of the secondary crRNA to the secondary Cas enzyme or prevents binding of the secondary crRNA/Cas complex to the activator. Each blocking moiety has a cleavable sequence configured to be cleaved by an activated Cas enzyme of an activated primary crRNA/Cas complex. Cleavage of the cleavable sequence of the blocking moiety releases the blocking moiety allowing the secondary crRNA to bind a secondary Cas enzyme and an activator to produce an activated secondary crRNA/Cas complex. The system also includes a plurality of probes, each probe having an oligonucleotide element labeled with a detectable label. The probe is configured to be cleaved by the activated secondary crRNA/Cas complex to generate a detectable signal or a detectable molecule.

In some embodiments, the primary Cas enzyme is a Cas12 enzyme and the secondary Cas enzyme is a Cas13 enzyme. In some such embodiments, the target to be detected is a ssDNA, dsDNA, or DNA/RNA heteroduplex sequence. In yet other embodiments target to be detected is an RNA sequence and the primary Cas enzyme is a Cas13 enzyme and the secondary Cas enzyme is a Cas12 enzyme. Other combinations of Cas enzymes are possible within embodiments of the present disclosure for optimizing detection of various targets.

The present disclosure also includes methods of detecting a target in a sample using a multiplexed CBTD system of the present disclosure.

Additional details regarding the methods, systems, and kits of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

Various Aspects and Embodiments of the Present Disclosure

The present disclosure further includes the following aspects and embodiments.

Aspect 1: A CRISPR/Cas chain reaction (CCR) system for amplifying the detection sensitivity of a primary CRISPR-based target detection (CBTD) system that comprises a plurality of CRISPR-associated (Cas) enzymes with activatable trans-cleavage activity and a plurality of primary CRISPR RNAs (crRNA) capable of forming a complex with one of the Cas enzymes to form a primary crRNA/Cas complex, each primary crRNA comprising a primary guide sequence configured to bind a target, such that binding of the primary crRNA/Cas complex to the target activates the trans-cleavage activity of the Cas enzyme to produce an activated primary crRNA/Cas complex, the CCR system comprising:
a plurality of secondary crRNAs capable of forming a complex with one of the Cas enzymes to form a secondary crRNA/Cas complex, each secondary crRNA comprising a secondary guide sequence configured to bind an activator;
a plurality of activators, each activator comprising an oligonucleotide element complementary to and configured to bind the secondary crRNA, such that binding of the secondary crRNA/Cas complex to the activator activates the trans-cleavage activity of the Cas enzyme to produce an activated secondary crRNA/Cas complex;
a plurality of blocking moieties bound either to the secondary crRNA or to the activator such that the bound blocking moiety prevents binding of the secondary crRNA to the Cas enzyme or prevents binding of the secondary crRNA/Cas complex to the activator, each blocking moiety comprising a cleavable sequence configured to be cleaved by an activated Cas enzyme of an activated primary crRNA/Cas complex or an activated secondary crRNA/Cas complex, such that cleavage of the cleavable sequence of the blocking moiety releases the blocking moiety allowing the secondary crRNA to bind another of the Cas enzymes and an activator to produce an activated secondary crRNA/Cas complex, resulting in a CRISPR/Cas chain reaction that produces additional activated secondary crRNA/Cas complexes; and
a plurality of probes, each probe comprising an oligonucleotide element labeled with a detectable label, wherein the probe is configured to be cleaved by any of the activated Cas enzymes of the activated primary crRNA/Cas complexes or the activated secondary crRNA/Cas complexes to generate a detectable signal or a detectable molecule, wherein the CCR system amplifies the detection sensitivity compared to the CRISPR-based target detection (CBTD) system alone.

Aspect 2: The CCR system of aspect 1, wherein the primary CBTD system comprises a commercially available CBTD system.

Aspect 3: The CCR system of aspect 1 or 2, wherein the blocking moiety is selected from: a nucleotide sequence configured to bind to the secondary crRNA or the activator that prevents binding of the secondary crRNA to the activator; a nucleotide sequence configured to bind to the secondary crRNA that prevents binding of the secondary crRNA to the Cas enzyme; and a large molecule or a surface linked to the secondary crRNA or the activator by a linking sequence that contains the cleavable sequence, such that the large molecule or surface sterically hinders binding of the secondary crRNA to the activator.

Aspect 4: The CCR system of aspect 3, wherein the blocking moiety is a blocking nucleotide sequence bound to the secondary crRNA that prevents binding of the secondary crRNA to the activator, wherein the blocking nucleotide sequence comprises one or more complementary segments bound to the secondary crRNA and one or more non-complementary, unbound segment having a sequence cleavable by an activated Cas enzyme, such that cleavage of the one or more non-complementary segments of the blocking nucleotide by the activated Cas enzyme releases the secondary crRNA from the blocking nucleotide such that the secondary crRNA can bind the activator.

Aspect 5: The CCR system of aspect 4, wherein at least one non-complementary, unbound segment occurs between at least two complementary segments, such that the non-complementary segment forms a bulge in the blocking nucleotide sequence.

Aspect 6: The CCR system of aspect 3, wherein the blocking moiety is a large molecule selected from: a protein; a lipid; a sugar; a nucleic acid; another large macromolecule; or a small molecule interacting with a magnetic particle, a nanoparticle, a peptide, a lipid, a sugar, a nucleic acid, or large macromolecule.

Aspect 7: The CCR system of aspect 3, wherein the blocking moiety is a surface, and wherein the and wherein the secondary crRNA or activator are covalently or non-covalently coupled to the surface b the linking sequence.

Aspect 8: The CCR system of any of aspects 1-7, wherein the target is a target polynucleotide sequence selected from: a ssDNA, a dsDNA, a ssRNA, a methylated DNA, a methylated RNA, or a heteroduplex of RNA and DNA.

Aspect 9: The CCR system of any of aspects 1-8, wherein the Cas enzyme is selected from Type V or Type VI Cas enzymes.

Aspect 10: The CCR system of aspect 7, wherein the Cas enzyme is selected from a Cas12 enzyme, a Cas13 enzyme, or a Cas14 enzyme.

Aspect 11: The CCR system of aspect 10, wherein the target is a ssDNA, a dsDNA, or a DNA/RNA heteroduplex sequence, and wherein the Cas12 enzyme is selected from: Cas 12a, Cas12b, Cas12c, Cas12d, Cas12e, Cas12f, Cas12g, Cas12h, Cas12i, Cas12j1-Cas12j10, or Cas12k.

Aspect 12: The CCR system of aspect 10, wherein the target is a ssDNA, a dsDNA, a or DNA/RNA heteroduplex sequence, and wherein the Cas14 enzyme is selected from: Cas14a1-Cas14a6, Cas14b1-Cas14b16, or Cas14c1-Cas14c2

Aspect 13: The CCR system of aspect 10, wherein the target is a ssRNA and the Cas12 enzyme is Cas12g.

Aspect 14: The CCR system of aspect 10, wherein the target is an RNA sequence and the Cas enzyme is a Cas13 enzyme selected from: Cas13a, Cas13b, Cas13c, or Cas13d.

Aspect 15: The CCR system of any of aspects 1-14, wherein the CCR system further comprises an excess of the Cas enzyme in addition to the Cas enzyme in the primary CBTD system.

Aspect 16: The CCR system of any of aspects 1-15, wherein the CCR system further comprises an excess of the primary crRNA in addition to the primary crRNA in the primary CBTD system.

Aspect 17: The CCR system of any of aspects 1-16, wherein the activators, secondary crRNAs, and probes are provided in excess.

Aspect 18: The CCR system of any of aspects 1-12 or 15-17, wherein the Cas enzyme is a DNA-targeting nuclease, and the cleavable sequence of the blocking moiety and the oligonucleotide element of the probe comprise a ssDNA sequence having at least 80% A and/or T.

Aspect 19: The CCR system of any of aspects 1-10 or 13-17, wherein the Cas enzyme is an RNA-targeting nuclease and the cleavable sequence of the blocking moiety and the oligonucleotide element of the probe comprise a ssRNA having at least 80% A and/or U.

Aspect 20: The CCR system of any of aspects 1-19, wherein the primary crRNA, the secondary crRNA, or both, comprise a polynucleotide extension sequence linked to a 3′-end of the guide sequence, the extension sequence having 1-31 nucleotides.

Aspect 21: The CCR system of aspect 20, wherein the polynucleotide extension sequence comprises a ssDNA or ssRNA.

Aspect 22: The CCR system of aspects 21, wherein the secondary crRNA comprises a polynucleotide extension sequence linked to a 3′-end of the guide sequence, wherein the blocking moiety is a nucleotide sequence configured to bind to the secondary crRNA and is linked to a 3′ end of the crRNA, and wherein the polynucleotide extension sequence is between the guide sequence and the blocking moiety or wherein the extension sequence comprises part of the blocking moiety.

Aspect 23: A CRISPR/Cas chain reaction (CCR) system for amplifying the detection sensitivity of a primary CRISPR-based target detection (CBTD) system that comprises a plurality of CRISPR-associated (Cas) enzymes with activatable trans cleavage activity and a plurality of primary CRISPR RNAs (crRNA) each comprising a primary guide sequence configured to bind a target and activate the primary CBTD system such that the primary crRNA and Cas enzyme form a primary crRNA/Cas complex that upon binding the target activates the trans-cleavage activity of the Cas enzyme to produce an activated primary crRNA/Cas complex, the CCR system comprising:
a plurality of secondary crRNAs capable of forming a complex with one of the Cas enzymes to form a secondary crRNA/Cas complex, each secondary crRNA comprising a secondary guide sequence configured to bind an activator;
a blocking nucleotide sequence bound to the secondary crRNA that prevents binding of the secondary crRNA to the Cas enzyme or the activator, wherein the blocking nucleotide sequence comprises one or more complementary segments bound to the secondary crRNA and one or more non-complementary, unbound segment having a sequence cleavable by an activated Cas enzyme of an activated primary crRNA/Cas complex or an activated secondary crRNA/Cas complex, such that cleavage of the one or more non-complementary segments of the blocking nucleotide releases the secondary crRNA from the blocking nucleotide such that the secondary crRNA can bind the activator and another of the Cas enzymes;
a plurality of activators, each activator comprising an oligonucleotide element complementary to and configured to bind the secondary crRNA, such that binding of the secondary crRNA/Cas complex to the activator activates the trans-cleavage activity of the Cas enzyme to produce an activated secondary crRNA/Cas complex, resulting in a CRISPR/Cas chain reaction that produces additional activated secondary crRNA/Cas complexes; and
a plurality of probes, each probe comprising an oligonucleotide element labeled with a detectable label, wherein the probe is configured to be cleaved by the activated Cas enzymes of the activated primary crRNA/Cas complexes or the activated secondary crRNA/Cas complexes to generate a detectable signal or a detectable molecule, wherein the CCR system amplifies the detection sensitivity compared to the CRISPR-based target detection (CBTD) system alone.

Aspect 24: The CCR system of aspect 23, wherein the blocking nucleotide sequence is connected to the secondary crRNA by a linker having a first end linked to a 3′ end of the secondary guide sequence and a second end linked to the blocking nucleotide sequence, such that the secondary guide sequence, linker, and blocking nucleotide sequence form a hairpin conformation, the linker having a sequence cleavable by the activated Cas enzyme, such that cleavage of the linker and any unbound segments of the blocking nucleotide sequence releases the secondary crRNA from the blocking nucleotide sequence.

Aspect 25: The CCR system of aspect 23 or 24, wherein at least one of the non-complementary, unbound segments occurs between at least two complementary segments, such that the non-complementary segment forms a bulge in the blocking nucleotide sequence.

Aspect 26: The CCR system of aspect 5 or aspect 25, wherein the one or more complementary segments are about 3-41 nucleotides long, and wherein the one or more non-complementary, unbound segment is from 2-40 nucleotides in length. According to some aspects, the total number of complementary nucleotides (e.g., combined complementary segments) of the blocking nucleotide sequence is about 3-44 nucleotides.

Aspect 27: A kit comprising the CCR system of any of aspects 1-26 and a primary CBTD system, the primary CBTD system comprising: a plurality of CRISPR-associated (Cas) enzymes with activatable trans cleavage activity and a plurality of primary CRISPR RNAs (crRNA) capable of forming a complex with one of the Cas enzymes to form a primary crRNA/Cas complex, each primary crRNA comprising a primary guide sequence configured to bind a target, such that binding of the primary crRNA/Cas complex to the target activates the trans-cleavage activity of the Cas enzyme to produce an activated primary crRNA/Cas complex.

Aspect 28: The kit of aspect 27, wherein the primary CBTD system comprises a commercially available CBTD system.

Aspect 29: The kit of aspect 27, wherein the primary crRNA comprises a polynucleotide extension sequence linked to a 3′-end of the guide sequence, and the polynucleotide extension sequence comprises a ssDNA or ssRNA having 1-31 nucleotides.

Aspect 30: The kit of aspect 29, wherein if the Cas enzyme is a DNA-targeting nuclease, the polynucleotide extension sequence comprises a ssDNA having a sequence of at least 80% A and/or T, but if the Cas enzyme is an RNA-targeting nuclease, the polynucleotide extension sequence comprises a ssRNA having a sequence of at least 80% A and/or U.

Aspect 31: The kit of any of aspects 27-30, wherein the kit comprises: an excess of the Cas enzyme, an excess of the primary crRNA, and an excess of activators.

Aspect 32: A method of amplifying the detection sensitivity of a primary CRISPR-based target detection (CBTD) system, the method comprising combining the primary CBTD system with a sample comprising a target to be detected and a CRISPR/Cas chain reaction (CCR) system of any of aspects 1-26, wherein the signal generated by the CCR system is greater than a signal produced from the primary CBTD system alone in the same amount of time.

Aspect 33: The method of aspect 32, wherein the target is not pre-amplified before combining with the primary CBTD system and/or the CCR system.

Aspect 34: The method of aspect 32 or 33, wherein the sample, the primary CBTD system and the CCR system are combined in a one-pot reaction.

Aspect 35: The method of any of aspects 32-34, performed at a temperature of about 20° C. to 75° C.

Aspect 36: The method of any of aspects 32-34, performed at about room temperature.

Aspect 37: The method of any of aspects 32-36, wherein the signal to be detected is selected from the group consisting of: fluorescence, luminescence, a chemical signal, a magnetic signal, a colorimetric signal, other optical signals, pH changes, temperature changes, electrochemical signals, and combinations of these.

Aspect 38: A multiplexed CRISPR-based target detection (CBTD) system comprising:
a primary CRISPR-based target detection (CBTD) system that comprises a plurality of primary CRISPR-associated (Cas) enzymes with activatable trans-cleavage activity and a plurality of primary CRISPR RNAs (crRNA) capable of forming a complex with one of the primary Cas enzymes to form a primary crRNA/Cas complex, each primary crRNA comprising a primary guide sequence configured to bind a target, such that binding of the primary crRNA/Cas complex to the target activates the trans-cleavage activity of the primary Cas enzyme to produce an activated primary crRNA/Cas complex; and
a secondary CBTD system that comprises:
   a plurality of secondary Cas enzymes with activatable trans cleavage activity, wherein the secondary Cas enzyme is different from the primary Cas enzyme;
   a plurality of secondary crRNAs capable of forming a complex with a secondary Cas enzyme to form a secondary crRNA/Cas complex, each comprising a secondary guide sequence configured to bind an activator;
   a plurality of activators, each activator comprising an oligonucleotide element complementary to and configured to bind the secondary crRNA, such that binding of the secondary crRNA/Cas complex to the activator activates the trans-cleavage activity of the secondary Cas enzyme to produce an activated secondary crRNA/Cas complex;
   a plurality of blocking moieties bound either to the secondary crRNA or to the activator such that the bound blocking moiety prevents binding of the secondary crRNA to the secondary Cas enzyme or prevents binding of the secondary crRNA/Cas complex to the activator, each blocking moiety comprising a cleavable sequence configured to be cleaved by an activated Cas enzyme of an activated primary crRNA/Cas complex, such that cleavage of the cleavable sequence of the blocking moiety releases the blocking moiety allowing the secondary crRNA to bind a secondary Cas enzymes and an activator to produce an activated secondary crRNA/Cas complex; and
   a plurality of probes, each probe comprising an oligonucleotide element labeled with a detectable label, wherein the probe is configured to be cleaved by the activated secondary crRNA/Cas complex to generate a detectable signal or a detectable molecule.

Aspect 39: The multiplexed CBTD system of aspect 38, wherein the primary Cas enzyme is a Cas12 enzyme and the secondary Cas enzyme is a Cas13 enzyme.

Aspect 40: The multiplexed CBTD system of aspect 39, wherein the target to be detected is a ssDNA, dsDNA, or DNA/RNA heteroduplex sequence.

Aspect 41: The multiplexed CBTD system of aspect 38, wherein the primary Cas enzyme is a Cas13 enzyme and the secondary Cas enzyme is a Cas12 enzyme.

Aspect 42: The multiplexed CBTD system of aspect 41, wherein the target to be detected is an RNA sequence.

Aspect 42: A CRISPR chain reaction (CCR) system comprising:
a secondary CRISPR/Cas system configured to become activated upon activation of a primary CBTD system that comprises a plurality of primary CRISPR RNAs (crRNA) having a primary guide sequence configured to bind a specific target and form a complex with the target and one of a plurality of CRISPR-associated (Cas) enzymes with activatable trans-cleavage activity to form an activated primary crRNA/Cas complex, wherein the plurality of Cas enzymes is part of the primary CBTD system, the CCR system, or both, the secondary CRISPR/Cas system comprising:
   a plurality of secondary crRNAs, each capable of forming a complex with one of the plurality of Cas enzymes to form a secondary crRNA/Cas complex, each secondary crRNA comprising a secondary guide sequence configured to bind an activator;
   a plurality of activators, each activator comprising an oligonucleotide element complementary to and configured to bind the secondary crRNA, such that binding of the secondary crRNA/Cas complex to the activator activates the trans-cleavage activity of the Cas enzyme to produce an activated secondary crRNA/Cas complex;
   a plurality of blocking moieties bound either to the secondary crRNA or to the activator such that the bound blocking moiety prevents binding of the secondary crRNA to the Cas enzyme or prevents binding of the secondary crRNA/Cas complex to the activator, each blocking moiety comprising a cleavable sequence configured to be cleaved by an activated Cas enzyme of an activated primary crRNA/Cas complex or an activated secondary crRNA/Cas complex, such that cleavage of the cleavable sequence of the blocking moiety releases the blocking moiety allowing the secondary crRNA to bind another of the Cas enzymes and an activator to produce an activated secondary crRNA/Cas complex, resulting in a CRISPR chain reaction that produces additional activated secondary crRNA/Cas complexes,
wherein each of the activated secondary crRNA/Cas complexes and each of the activated primary crRNA/Cas complexes is capable of cleaving a plurality of probes included in the primary CBTD system, the CCR system, or both, each probe comprising an oligonucleotide element labeled with a detectable label, the oligonucleotide element configured to be cleaved by any of the activated primary crRNA/Cas complexes or the activated secondary crRNA/Cas complexes to generate a detectable signal or a detectable molecule, wherein the CCR system amplifies the detection sensitivity compared to the primary CBTD system alone.

Additional aspects include a CCR system according to aspect 42 including any of the embodiments of aspects 2-22 or 24-26.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1—Amplification-Free Nucleic Acid Detection of Targets at Room Temperature Using CRISPR/Cas Chain Reaction

Introduction

The present example describes development and testing of embodiments of a CRISPR/Cas based amplification-free diagnostic method and system called CRISPR/Cas Chain Reaction (CCR) such as describe above and illustrated in FIG. 1 and FIGS. 2A-2C and FIG. 3. This method/system combines a primary, on-target CRISPR/Cas system with a ‘locked’ secondary CRISPR/Cas system (also referred to as CRISPR-AMP system or CCR system) that includes a modified crRNA (also called the “CRISPR-AMP” crRNA or “secondary crRNA) that is locked for activity. The locked secondary system becomes unlocked and produces an enhanced signal only after the primary CRISPR/Cas system detects its target and initiates non-specific collateral (trans) cleavage, which then activates the secondary system which, once activated, can then self-activate additional secondary crRNA's as well as reporters. In the embodiment described in this example, combining the CCR platform with reverse transcriptase (if needed to convert RNA target to DNA) provided amplification-free detection of attomolar levels of a wide variety of synthetic DNA and RNA targets including Malaria, HIV-1, and SARS-CoV-2 within 60-90 minutes at room temperature. A significant advantage of the described CCR platform is that it is a universal CRISPR/Cas amplification system and can be rapidly coupled with any CRISPR-based target detection (CBTD) system to enhance its sensitivity of detection.

Most Class 2 CRISPR/Cas systems, including CRISPR/Cas12 (Type V), CRISPR/Cas14 (Type V), and CRISPR/Cas 13 (Type VI) mediate a nonspecific collateral trans-cleavage of random DNA and RNA after binding or cis-cleavage of their target DNA or RNA. For CRISPR/Cas12a, this multiple-turnover trans-cleavage activity is only initiated once the crRNA/Cas12a complex is bound to its target ssDNA or dsDNA that acts as an activator. This trans-cleavage activity has been widely exploited for nucleic acid detection and has been combined with fluorescence-based, paper-based, and electrochemical-based sensing technologies to develop rapid and sensitive diagnostics. However, current CRISPR/Cas12a systems are limited to a nanomolar detection limit unless the target is either pre-amplified in some manner or advanced detection methods are employed.

In previous experiments, crRNAs with different end modifications were then tested in order to better understand and modulate the trans-cleavage activity, which resulting in improved sensitivity and specificity of detection. Then, it was discovered that certain extensions and modifications to crRNA could potentially change its nature of binding and subsequently alter this trans-cleavage due to conformational changes of the Cas12a dynamic endonuclease domain.

By extending the 3′- or 5-ends of the crRNA with different lengths of DNA, RNA, and phosphorothioate DNA, a new self-catalytic behavior was discovered, and also an augmented rate of Cas12a-mediated trans-cleavage activity as high as 3.5-fold compared to the wild type crRNA. This reflected an unprecedented improvement in sensitivity and limit of detection of nucleic acid targets down to the femtomolar level. This new, highly sensitive system could be used to detect as low as 25 f M dsDNA from PCA3, an overexpressed biomarker in prostate cancer patients, in simulated urine in 6 hours without target pre-amplification. The same platform was determined to detect as low as ˜700 fM ssDNA from human immunodeficiency virus (HIV), and 290 fM RNA from Hepatitis C virus (HCV) in a buffer within thirty minutes without any target amplification. Furthermore, several crRNA extensions were discovered within the 3′-end of the crRNA that also improved the specificity of detection discriminating single nucleotide differences. These design principles and strategies can be extended to improve the activity and specificity of other variants of CRISPR/Cas.

However, even these enhanced CRISPR-based detection methods “CRISPR-ENHANCE” (described in greater detail in PCT application PCT/US2020/059577 (publication WO 2021/092519) which is hereby incorporated herein by reference in its entirety), often employ an additional amplification step using an isothermal recombinase polymerase amplification (RPA) to detect target at the lowest levels, To allow even lower level target detection without the DNA amplification step, the present disclosure presents an amplified CRISPR/Cas system, “CRISPR-AMP” that employs both the enhanced CRISPR/Cas detection described in greater detail below along with a second CRISPR/Cas that is inactivated until activation by the enhanced CRISPR/Cas upon detection of target, providing a CRISPR/Cas chain reaction (CCR). In the present disclosure, the CCR system is also sometimes referred to as CRISPR-AMP.

Type V and VI CRISPR/Cas systems are generally used for detecting a target DNA or RNA because once they find their specific target they turn on a collateral cleavage activity for single-stranded DNA or RNA reporters, that can then produce a fluorescence signal after cleavage. Normally 1-10 nM concentrations of a target can be easily detected by these systems. As discussed above, an embodiment of our CRISPR-ENHANCE technology includes a 7-nucleotide DNA modification on the 3′ end of crRNAs that enhances the collateral cleavage activity by 3.5 fold, which allows for high fM detection of targets within 30 minutes. However, it was found that certain longer extensions to crRNA including DNA extensions >19-nt and phosphorothioate extensions >13-nt can completely inactivate the CRISPR/Cas activity.

From these observations, a new technology was developed combining two sets of CRISPR/Cas complexes where one set of CRISPR/Cas complexes is in the active form and the other set is in the inactive form (see FIG. 2B and FIG. 3). The active set of CRISPR/Cas can find and detect a low concentration of the target such as 1 aM concentration or a few copies of the target. This then initiates a trans cleavage (FIG. 2 B, step 4, and FIG. 3, step (c)), which activates a secondary CRISPR/Cas complex which can bind with excess of synthetic/secondary target (“activator”) added in the mixture (FIG. 2B, step 4′, and FIG. 3, step (d)). This cascade of CRISPR/Cas activation turns on excess of trans cleavage activity which can then cleave additional locked secondary CRISPR/Cas complex as well as cleave and and “turn on” an excess of reporter within minutes (FIG. 2B, step 4′, and FIG. 3, step (e)). This secondary activation produces exponential amplification of reporters within minutes allowing the detection of low aM concentrations of the target without any target pre-amplification.

Such a system can be performed at room temperature, which will be groundbreaking for detecting various targets including SARS-CoV-2. The system will also reduce the cost and remove an additional RT-LAMP step. The system is highly sensitive as well as specific. In embodiments, everything can be mixed in a single pot a single step test. The detection can be done using fluorescence measurement, fluorescence visualization, paper-based test, electrochemical test, or other platforms. The system can be combined with lateral flow assays such as illustrated in FIG. 2C for easy point-of-care detection and diagnosis.

The system can be tailored to be activated in the presence of a DNA or RNA target and can be extended to a small molecule or protein target. The system can be optimized with various blocking mechanisms for “locking” the secondary CRISPR/Cas system, some of which are illustrated in FIG. 4. Various CRISPR/Cas systems can be combined and multiplexed. All these systems can be further coupled with an enzyme amplification system where an aptamer binds to a target and activates the CRISPR/Cas, while the CRISPR/Cas activation results in the activation of a second enzyme that can then cause a faster catalytic event that can be measured. Such a system can be developed by tethering an amplification enzyme via a nucleic acid linker that can be broken up upon the CRISPR/Cas activation. All these systems can be incorporated with a variety of reporter systems based on either or combination of fluorescence, luminescence, color change, product formation, redox reaction, pH change, surface reaction or cleavage, change in electrical conductivity, resistance, or impedance.

Additional description of the CRISPR-AMP/CCR system is provided in the attached figures and discussion below. This CCR system can be combined with other primary CRISPR/Cas systems for low level detection without the need for a PCR or RT-LAMP amplification step.

### Materials and Methods

### For all Experiments:

Reagents: All DNA/RNA oligonucleotides including all crRNAs, activators, uDNAs, uRNAs, hairpin blocker RNAs and DNAs, synthetic DNA and RNA targets, and Fluorophore-quencher reporters were ordered from Integrated DNA Technologies (IDT). Buffer NEB2.1, dNTPs, RNase inhibitor and the enzyme Reverse Transcriptase were ordered from New England Biolabs (NEB). All Cas enzymes including LbCas12a and different Cas13 enzymes were expressed and purified in-house.

Fluorescence measurements in CCR: All fluorescence measurements were carried out in a 384-well reaction plate using either a Synergy BioTek or Synergy Neo microplate reader. The fluorophore-quencher reporter molecule was excited at 485 nm and its emission at 528 nm was recorder using the microplate reader.

CRISPR/Cas Chain Reaction Assay: The CCR assay is performed as follows. First, a mastermix is prepared by mixing the primary crRNA, the primary Cas enzyme, 1×NEB2.1 buffer, GFP-Activator and DEPC-treated water. This mastermix is incubated for 10 mins at either room temperature or 37 C (or other temperature if indicated) based on the experiment. Then, the primary target and the blocked secondary crRNA are added to the mix and it is further incubated at 37 C for 20 min. Finally, the mastermix is added to a 384-well plate containing 500 nM of the FQ reporter. The 384-well plate is incubated in a microplate reader and measurements are taken every 2.5 mins. The concentrations of the different components vary by experiments and are indicated in the figure descriptions below.

Unlinked DNA/RNA blockers (uDNA/uRNA): For embodiments of the CCR systems of the present disclosure in which the blockers are secondary crRNA blockers that are separate and not linked to the crRNA using the hairpin loop (called uDNAs) as shown in FIG. 5, the secondary crRNA blockers were created using the following protocol. An unblocked crRNA and its complementary uDNA molecules of different lengths were mixed together in a 1:1 molar ratio and annealed by heating at 95° C. for 4 min, followed by gradient cooling to 25° C. at a rate of 0.1° C./s.

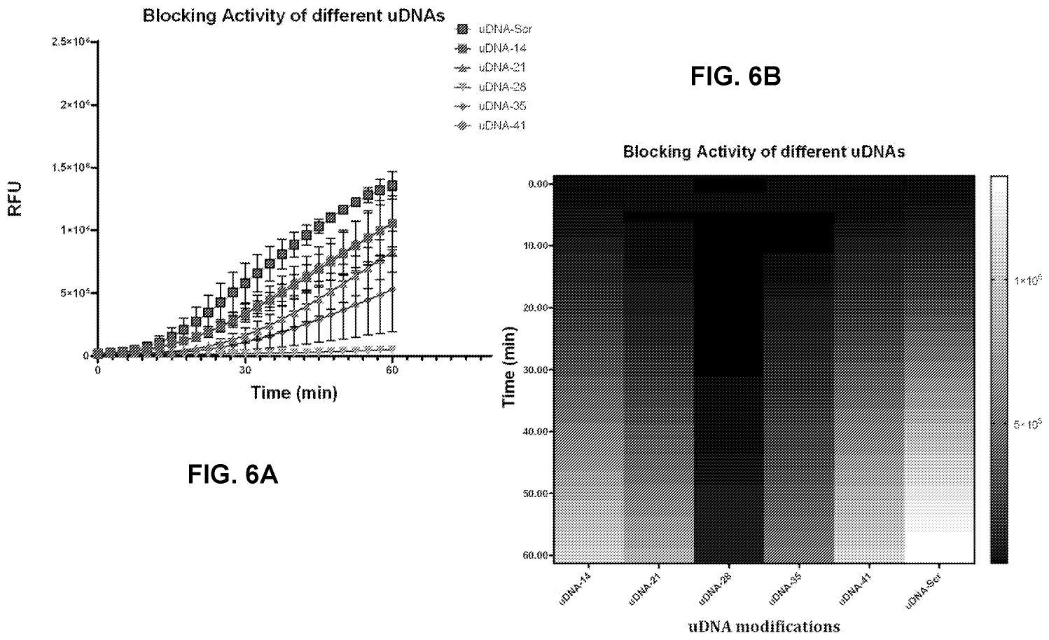

Assays for the blocking activity for the uDNA annealed crRNAs: To measure the blocking activity of bound uDNAs (uDNA14-uDNA41) (FIGS. 6A-6B) the following protocol was used. An unblocked crRNA that was annealed with a scrambled, non-complementary uDNA that does not block it (uDNA-Scr) was used as a control. 50 nM LbCas12a, 50 nM of the different uDNA-crRNAs, 50 nM of the target GFP-activator and 500 nM of FQ reporter (/56-FAM/TTATT/3IABkFQ) were mixed together and incubated at 37° C. for 1 hr. Fluorescence measurements were done every 2.5 mins during the incubation period using a Synergy BioTek microplate reader.

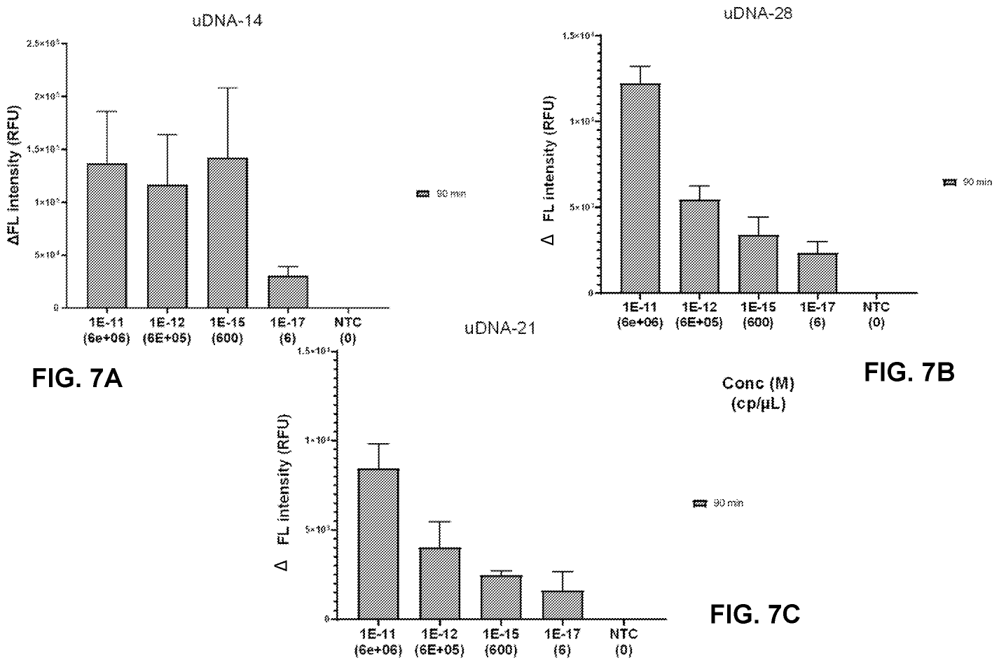

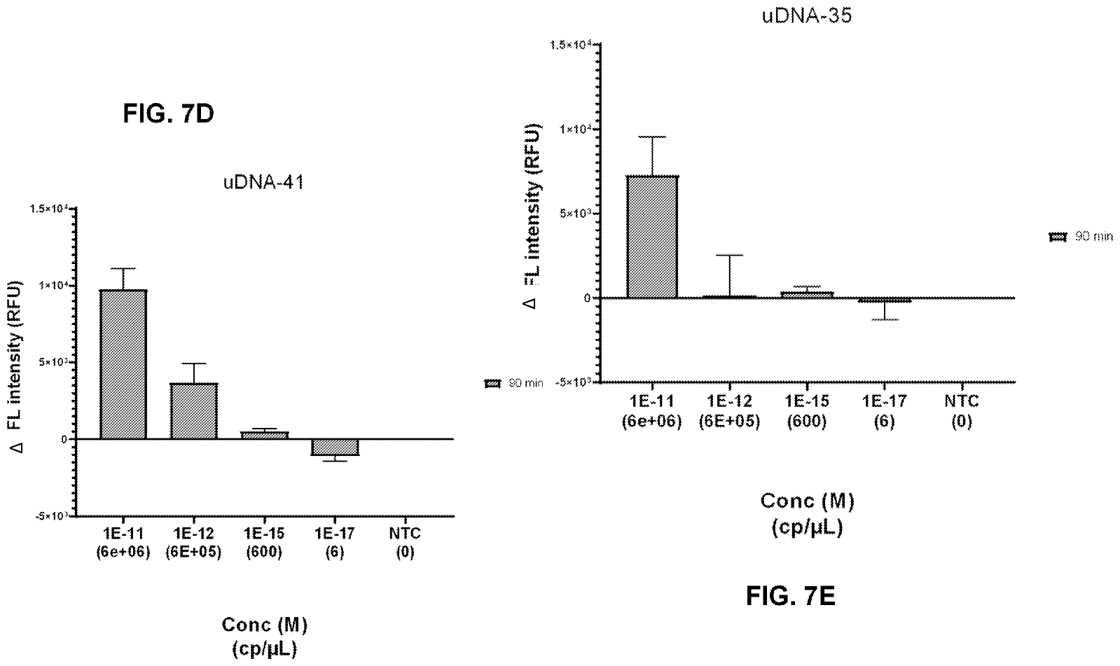

HIV detection with CRISPR/Cas Chain Reaction: The CCR system including crRNAs annealed with different uDNAs were used to detect several different dilutions of a synthetic HIV target DNA as shown in FIGS. 7A-E. The detection assay included 30 nM of the HIV targeting crRNA (crHIV), 60 nM LbCas12a, 60 nM uDNA-annealed crRNA that targets the GFP-activator, 500 nM FAM-FQ reporter (/56-FAM/TTATT/3IABkFQ/) and 10 nM GFP-Activator. Fluorescence measurements were taken every 2.5 minutes while incubating all the components together at 37° C. Background subtracted fluorescence intensities at t=90 min are plotted.

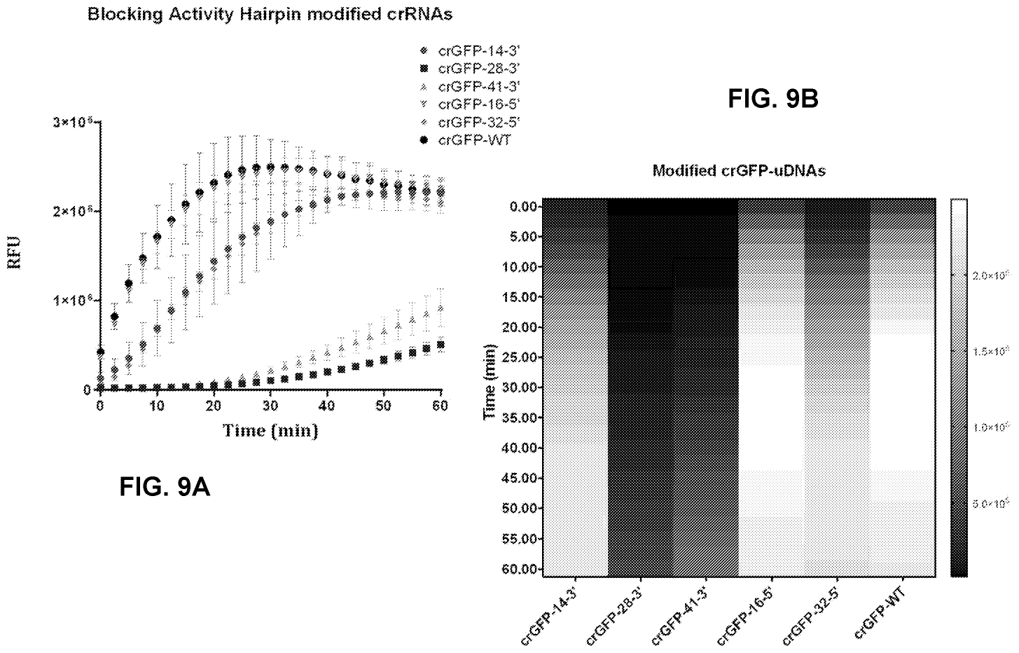

Assays for the blocking activity of the crRNAs with bound blocker: This assay was to test crRNA's that have the blocking DNA conjugated to them through a hairpin loop at the 3′-end or the 5′-end (FIG. 8). These crRNAs target a small double-stranded GFP activator. crGFP-WT represents the Wild Type crRNA that does not contain any hairpin loop or blocking DNA and is used as a control. The activity of the 5 different hairpin loop modified crRNAs and the wild-type control was measured by incubating 50 nM crRNA, 50 nM LbCas12a, 50 nM GFP-activator and 500 nM of FQ reporter (/56-FAM/TTATT/3IABkFQ) at 37° C. (FIGS. 9A-9B). Fluorescent measurements were done every 2.5 mins using a Synergy BioTek microplate reader.

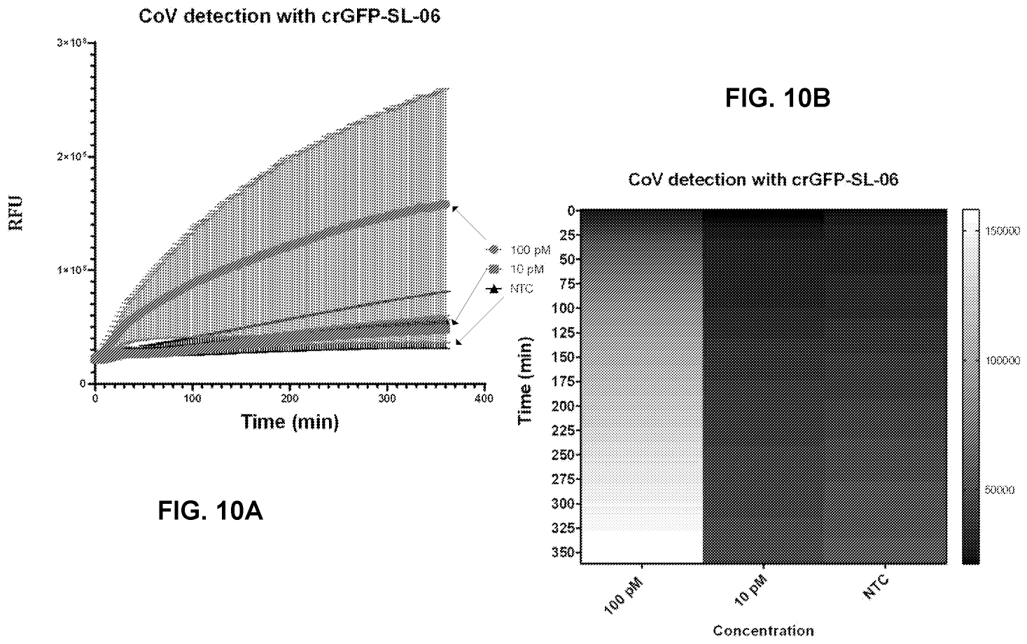

Assays for self-blocking hairpin loop modified crRNA: These experiments were performed with a different designs of the hairpin loop modified crRNA. Unlike the crRNAs with bound blockers shown in FIG. 8, the crRNA's used in these assays do not have the 7 nt non-complementary sections/bulges. They only have the noncomplementary hairpin loop sections. For these assays (results shown in FIGS. 10A-10B) 100 pM and 100 fM of a synthetic SARS-CoV-2 target (CoV) were detected using the CRISPR/Cas Chain Reaction assay using the self-blocking hairpin loop modified crRNA that targets a short double-stranded GFP activator. Different concentrations of the CoV target were incubated with 30 nM crCoV, 30 nM crGFP-magnetic, 120 nM LbCas12a, 10 nM GFP activator and 500 nM FAM-FQ reporter (/56-FAM/TTATT/3IABkFQ/). Fluorescence measurements were done every 2.5 minutes at 37° C.

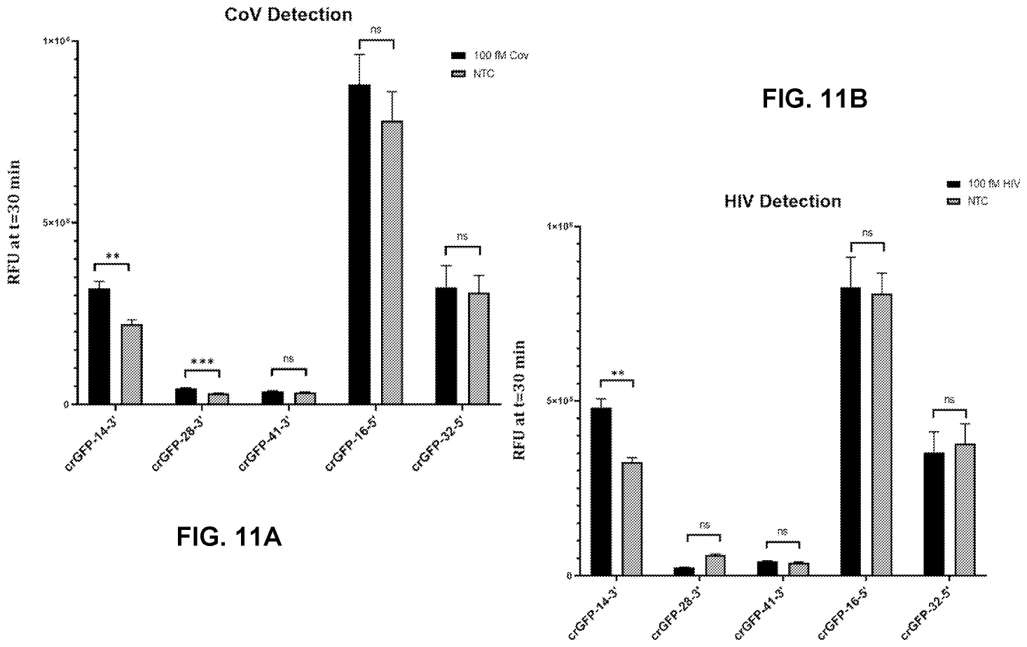

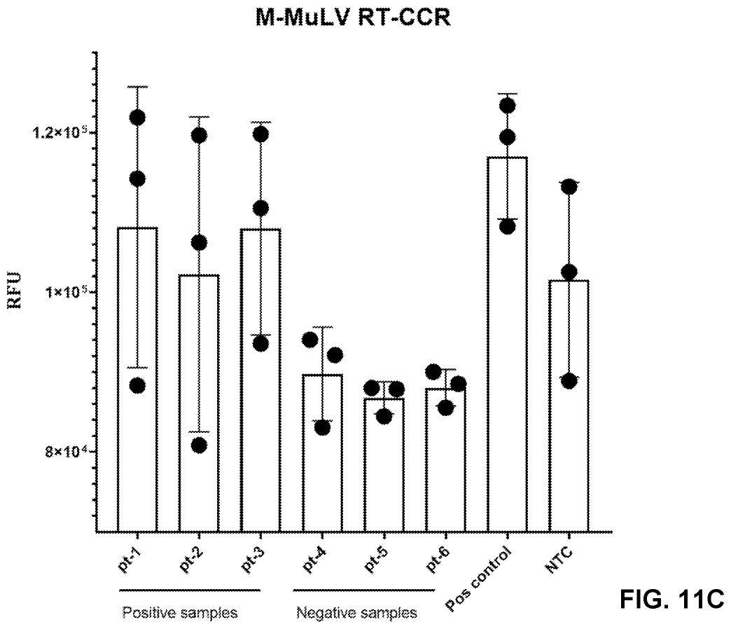

Detection of SARS-CoV-2 and HIV targets with CCR: To test detection of viral polynucleotide targets, hairpin modified blocked crRNAs for CRISPR/Cas Chain Reaction were tested (FIGS. 11A-11B). The 5 different hairpin modified crRNAs illustrated in FIG. 8 were used to detect 100 fM of a synthetic SARS-CoV-2 (CoV) or HIV target. The detection assay included 30 nM crHIV or crCoV, 60 nM LbCas12a, 60 nM of various hairpin-modified crGFP, 500 nM FAM-FQ reporter (/56-FAM/TTATT/3IABkFQ/) and 10 nM GFP-Activator. Fluorescence measurements were taken every 2.5 mins. Plot of Mean Raw Fluorescence Unit (RFU) for n=3 replicates at t=30 min is shown. NTC=No Target Control.

CCR detection of SARS-CoV-2 in patient samples: Six different patient samples were tested for SARS-CoV-2 RNA (3 pos, 3 neg as pre-determined by qPCR) by combining a Reverse Transcription step with the CCR assay. M-MuLV reverse transcriptase was used according to manufacturer's protocol. The reverse transcribed samples were subjected to the CCR assay including 50 nM LbCas12a, 50 nM crGFP-14-3′ (hairpin modified blocked crRNA), 40 nM crCoV and 5 nM GFP-activator. Genomic RNA from a Heat inactivated SARS-CoV-2 isolate was diluted to 1000 cp/uL was spiked in a healthy patient sample and used as the positive control. Similarly, DEPC-treated water was spiked in a healthy patient sample and used as the NTC. The CCR reaction was incubated at 37° C. and fluorescence measurements were taken every 2.5 mins. RFU at t=22.5 min is indicated with results shown in FIG. 11C. Error bars represent SD (n=3). Each individual data point is represented by a black dot.

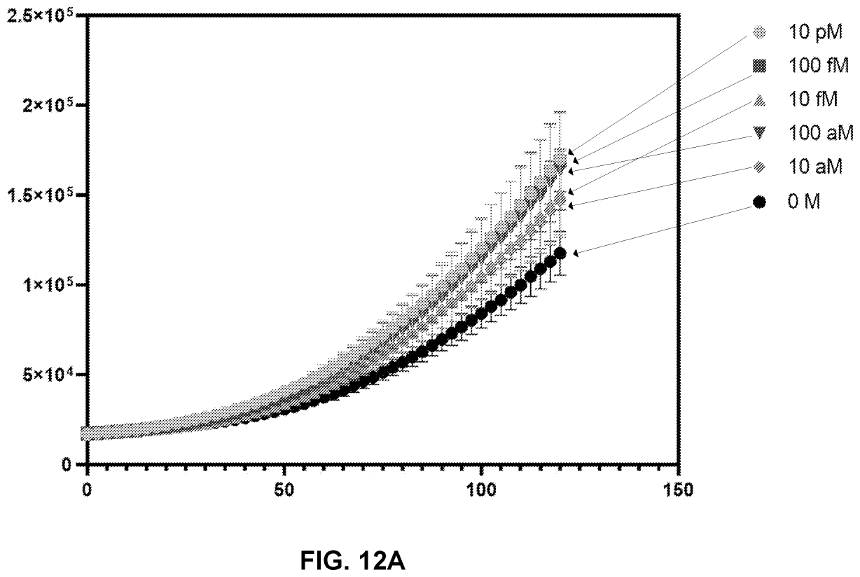

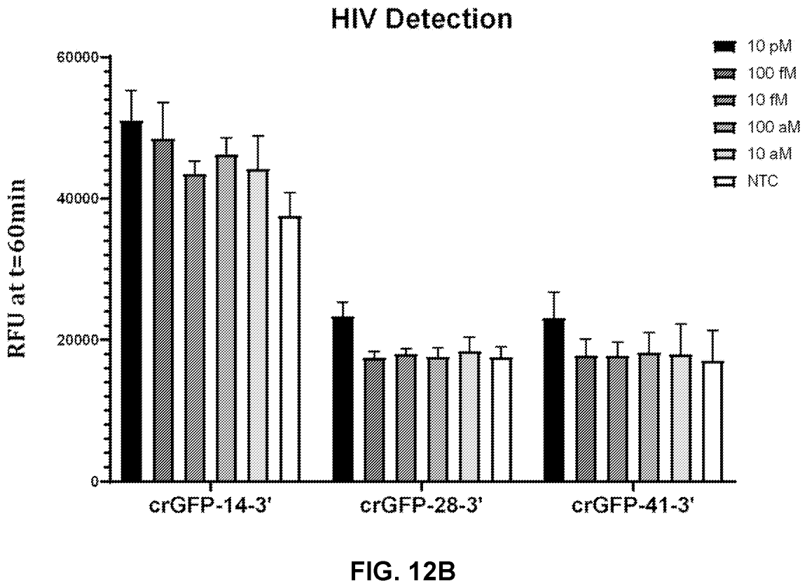

Detection of different target concentrations: Several different dilutions of a synthetic HIV target were tested using different hairpin loop blocked crRNAs in order to assay the limit of detection (LoD) of the different modified crRNAs (FIGS. 12A-12B). 50 nM of the hairpin modified crGFP-14-3′, crGFP-28-3′ and crGFP-41-3′ were used to detect 10 pM-10 aM dilutions of a synthetic HIV DNA. 50 nM LbCas12a, 50 nM of the different crGFPs, 10 nM GFP-Activator and 500 nM FAM-FQ reporter (/56-FAM/TTATT/3IABkFQ/) were used in the reaction. The fluorescent measurements were taken at 37° C.

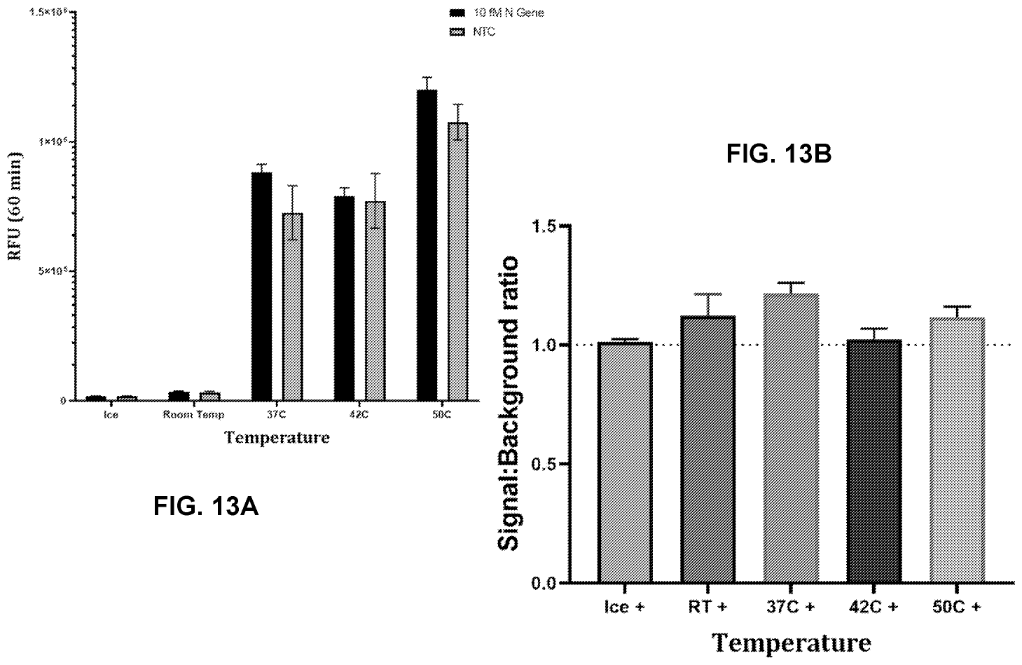

CCR at different temperatures: This experiment was done to test the effect of temperature on CRISPR/Cas Chain Reaction. For the experiments shown in FIGS. 13A-13B, 10 fM of a synthetic DNA resembling the N-gene of SARS-CoV-2 was incubated at the indicated temperatures with 25 nM of the N-gene targeting crN2, 50 nM LbCas12a, 50 nM crGFP-14-3′ and 500 nM of FAM-FQ reporter (/56-FAM/TTATT/3IABkFQ/). A single-point fluorescence measurement was done at 25° C. after 60 min of incubation at the different temperatures. RFU of the single-point reading done after 60 min incubation. Plot of Signal:Noise ratio at each different temperature was calculated by taking the ratio of the mean RFUs of 10 fM N gene target and the No Target Control (NTC). Higher signal:noise ratio indicates better detection.

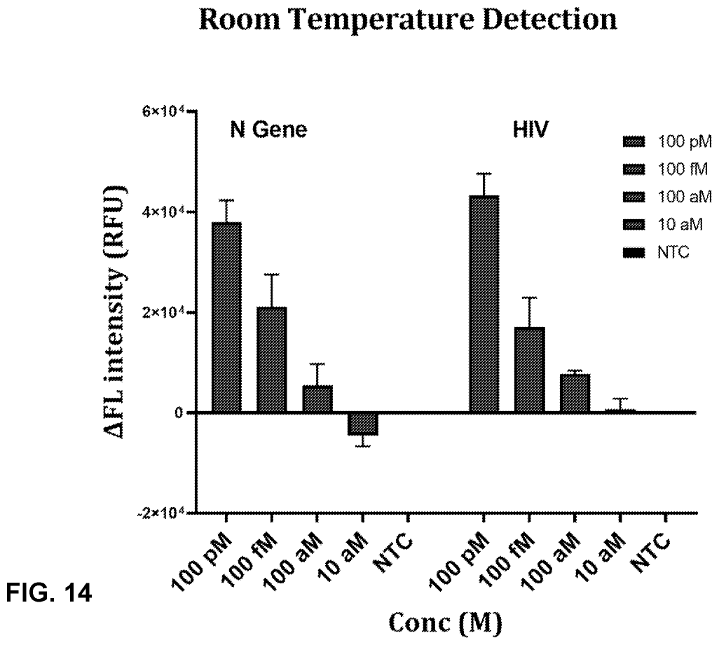

Room Temperature detection with CCR: Different concentrations of a synthetic CoV and HIV targets were tested at room temperature using CRISPR/Cas Chain Reaction. 50 nM of the hairpin modified blocked crGFP-14-3′ were used to detect 100 pM-10 aM dilutions of a synthetic HIV and SARS-CoV-2 N gene DNA. 50 nM LbCas12a, 50 nM of the crGFP-14, 10 nM GFP-Activator and 500 nM FAM-FQ reporter (/56-FAM/TTATT/3IABkFQ/) were used in the reaction. The fluorescent measurements were taken at 25° C. and results shown in FIG. 14. Background subtracted RFU at t=20 min is indicated.

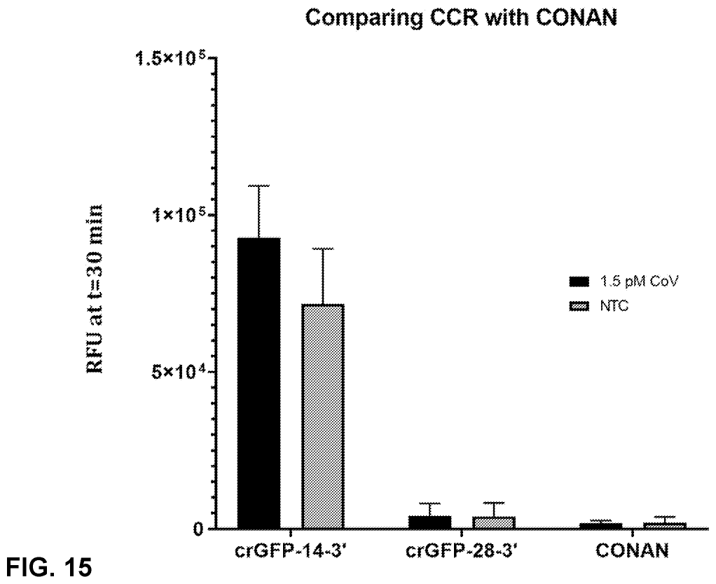

Comparing CRISPR/Cas Chain Reaction (CCR) with CONAN: In this experiment, the performance of an embodiment of CCR detection of a synthetic SARS-CoV-2 gene using the hairpin modified blocked crGFP-14-3′ and crGFP-28-3′ was compared against the previously published method called “CONAN” (Shi K, Xie S, Tian R, et al., Sci Adv. 2021). 1.5 pM of a synthetic SARS-CoV-2 gene was incubated with 50 nM crCoV, 100 nM LbCas12a and 50 nM of either crGFP-14-3′, crGFP-28-3′ or GFP-targeting scgRNA construct based on “CONAN.” 5 nM of double-stranded GFP activator and 500 nM of FAM-FQ reporter (/56-FAM/TTATT/3IABkFQ/) was used in the assay. Fluorescence measurements were taken every 2.5 mins at 37° C. using a Synergy BioTek microplate reader. Mean RFU (n=3) at time=30 min is plotted for each construct (FIG. 15). Error bars represent SD.

### Results/Discussion

The data presented in FIGS. 6A and 6B illustrates that 5 uDNAs of different length (illustrated in FIG. 5) were able to inhibit the native trans-cleavage activity of CRISPR/Cas by blocking the crRNA. uDNA-Scr, which is the unblocked crRNA used as a control, had the highest trans-cleavage activity. However, uDNA-14, uDNA-21, uDNA-28 and uDNA-41 all reduced the trans-cleavage activity of the crRNA to various degrees. uDNA-41 showed the least amount of blocking whereas uDNA-28 showed the highest blocking.

Then five of the uDNA-blocked crRNAs were used in an embodiment of a CCR system of the present disclosure to detect HIV. The results demonstrated that the different uDNA blocked crRNAs are able to detect the HIV target at very low concentrations (FIGS. 7A-7E). In all cases, the data is background subtracted in a way as to make the average fluorescence intensity of the No Target Control (NTC) reduce to 0. All fluorescence intensity measurements >0 in the bar plots represent positive detection of the target.

Then, 5 different blocking moieties were designed and tested, in which the blocking moiety was a single stranded DNA conjugated to the respective crRNAs through a cleavable hairpin loop at the 3′-end or 5′-end (illustrations of the 5 different hairpin modified blocked crRNAs are illustrated in FIG. 8). As demonstrated in FIGS. 9A-9B, the hairpin modified blocked crRNAs were also able to diminish the trans-cleavage activity of the CRISPR/Cas system by blocking the crRNA.

A different version of hairpin modified blocked crRNA without any bulges was also tested for detection of a short double-stranded GFP activator. As shown in FIGS. 10A-10B, these bulge-less variations of blocked crRNA were also able to detect 100 pM concentration of a synthetic SARS-CoV-2 target.

The 5 different hairpin modified crRNAs illustrated in FIG. 8 were designed used to detect 100 fM or CoV or HIV target. Positive detection occurs when target shows a higher fluorescence intensity than the No Target Control (NTC). As illustrated in FIGS. 11A-11B, CrGFP-14-3′ was robustly able to detect both the HIV and CoV target.

To test the use of CCR to detect target in patient samples, 6 different patient samples for SARS-CoV-2 (3 pos, 3 neg as determined by qPCR) were tested using the crGFP-12-3′ as the secondary crRNA with attached blocker (modified hairpin variation). Reverse transcription was performed on samples to convert RNA to DNA prior to the CCR assay. As shown in FIG. 11C, positive samples were distinguishable from the negative samples.

The CCR system was tested with different concentrations of HIV. All concentrations having a higher fluorescence signal than the NTC were considered positive for detection. As shown in FIGS. 12A-12B, crGFP-14-3′ was able to detect 10 attomolar (aM) or higher of the HIV target. CrGFP-28-3′ and crGFP-41-3′ were able to detect 10 pM of the HIV target but could not detect lower concentrations.

The CCR systems were then tested at different temperatures for detection of a DNA resembling the N-gene of SARS-CoV-2. Data shown in FIG. 13A indicated that the overall trans-cleavage activity was much higher at higher temperatures (37 C, 42 C and 50 C) as compared to room temperature or on ice. However, the signal: background ratio at room temperature (FIG. 13B) was similar to that at 37 C indicating that CCR can potentially be done at room temperature. FIG. 14 also demonstrates detection of various concentrations of either HIV or a DNA resembling the N-gene of SARS-CoV-2 using a CCR system of the present disclosure. The results demonstrate that CCR can be used to detect low concentrations of different targets at room temperature. Positive detection occurs when Fluorescence intensity is higher than that of the NTC. As shown, 100 aM or higher concentrations of the N gene or HIV were detected using crGFP-14-3′ hairpin modified crRNA. However, 10 aM concentration was not robustly detected for either gene. Different Cas enzymes also have been shown to have differing activity levels at different temperatures. Thus, the design of crRNAs for use with specific Cas enzymes can be used to optimize detection at higher or lower temperatures.

To compare an embodiment of the CCR system of the present disclosure to a recently published detection method called “CONAN,” the two detection systems were compared for detection of a synthetic CoV target structure. Data shown in FIG. 15 indicates that crGFP-14-3′ was able to distinguish between 1.5 pM CoV target and NTC much faster than “CONAN.”

### Example 2—CRISPR/Cas Chain Reaction Systems with Different Blocking Mechanisms

For the present example, general experimental materials and methods are same as those provided above in Example 1, except for the design of the blocking moiety. Variations of blocking mechanism are illustrated in FIG. 16 showing different approaches for modification of the secondary crRNA.

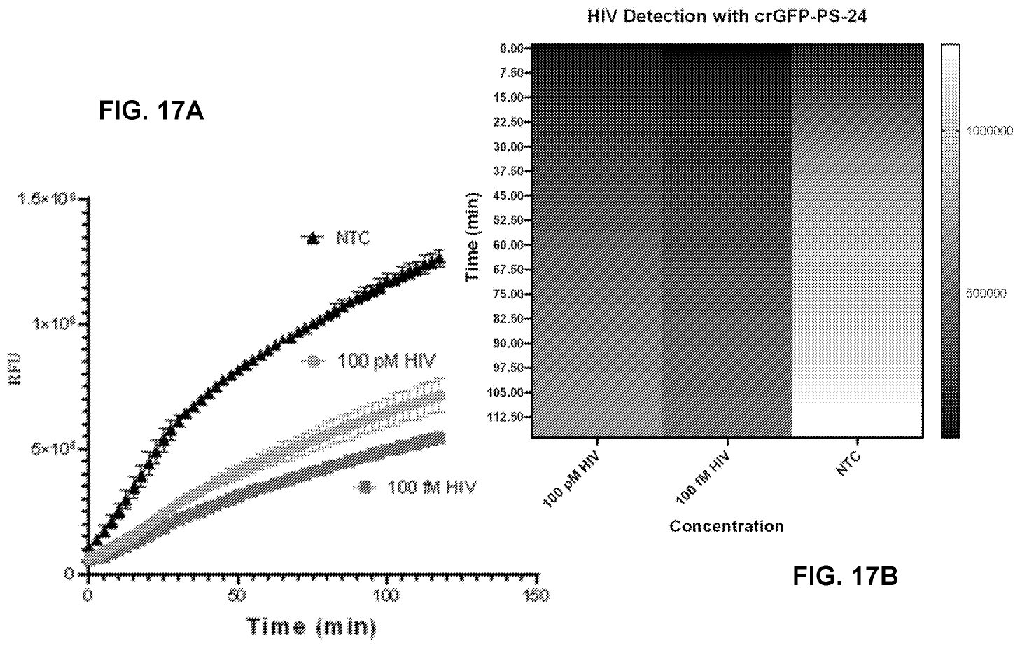

In an experiment to test phosphorothioate modified crRNA (such as illustrated in FIG. 16, model (a), 100 pM and 100 fM of a synthetic HIV target were detected using the CCR assay using the 24-mer phosphorothioate modified crRNA that targets a short double-stranded GFP activator. Different concentrations of HIV target were incubated with 30 nM crHIV, 30 nM crGFP-PS24, 120 nM LbCas12a, 10 nM GFP activator and 500 nM FAM-FQ reporter (/56-FAM/TTATT/3IABkFQ/). Fluorescence measurements were done every 2.5 minutes at 37° C. FIG. 17A is a plot of Mean RFU (n=3) w.r.t time is shown. Error bars represent SD. FIG. 17B shows a heat map of the data in FIG. 17A. This experiment demonstrates the performance of the phosphorothioate-modified crRNA for detection of 100 pM and 100 fM of a synthetic HIV DNA target. Positive detection occurs when the fluorescence intensity is higher than the intensity of the NTC. In this experiment, as the NTC has the highest intensity, neither of the concentrations of the target were detected.

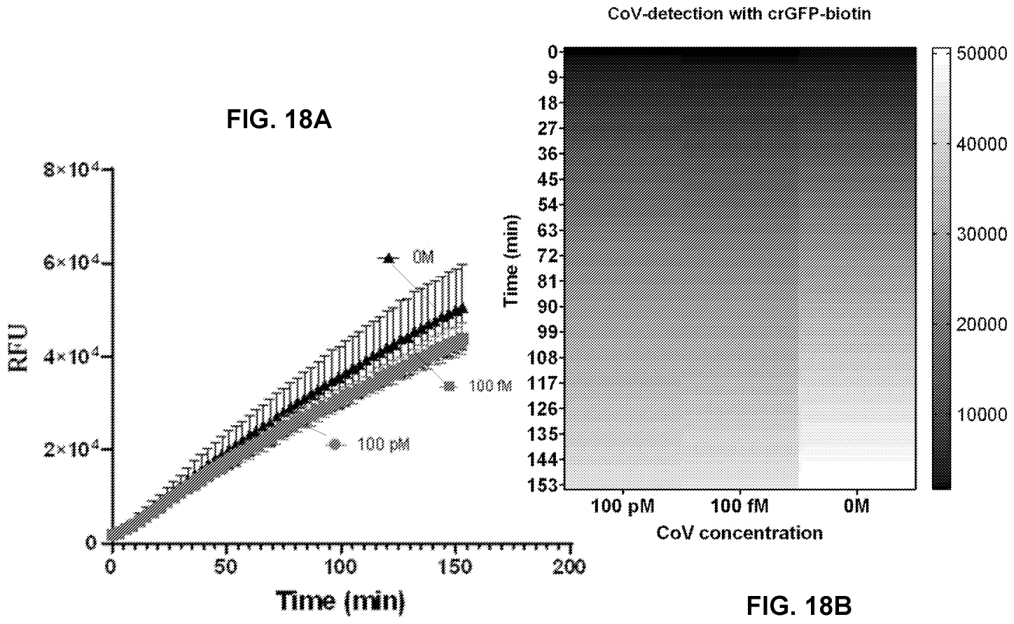

In an experiment to test crRNA modified by non-covalent coupling to a magnetic bead crRNA (such as illustrated in FIG. 16, model (e), streptavidin coated Dynabeads were non-covalently coupled to biotinylated crRNAs following manufacturer's protocol. 100 pM and 100 fM of a synthetic SARS-CoV-2 target were detected using the CCR assay using the magnetic bead-biotin modified crRNA that targets a short double-stranded GFP activator. Different concentrations of the CoV target were incubated with 30 nM crCoV, 30 nM crGFP-magnetic, 120 nM LbCas12a, 10 nM GFP activator and 500 nM FAM-FQ reporter (/56-FAM/TTATT/3IABkFQ/). Fluorescence measurements were done every 2.5 minutes at 37° C. FIG. 18A is a plot of Mean RFU (n=2) w.r.t time is shown. Error bars represent SD. FIG. 18B is a heat map of the data in FIG. 18A.

### Example 3—Multiplexed Orthogonal CRISPR/Cas Detection Systems

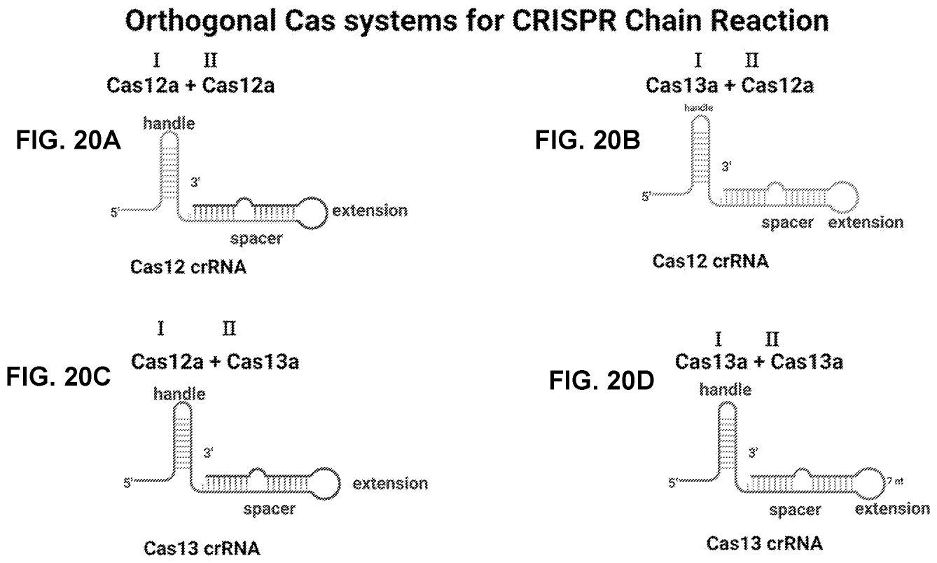

As described above, the present disclosure also provides multiplexed CRISPR/Cas based target detection systems that instead of a primary CBTD system and a CCR system employ a primary and secondary CBTD system with orthogonal CRISPR/Cas systems. Such systems multiplex different orthologs of crRNA and Cas enzymes to optimize detection of certain targets. The present example describes such a multiplexed orthogonal system that uses a combination of a Cas12a and a Cas13a system. FIGS. 20A and 20D illustrate two CRR systems where the primary and secondary crRNA are of the same type and the same Cas enzyme can be used for the whole system (e.g., Cas12a primary and secondary crRNA and Cas12a Cas enzyme (FIG. 20A), or Cas13a primary and secondary crRNA and Cas13a Cas enzyme (FIG. 20B)). In such systems the probe is also configured to be cleaved by the same type of Cas enzyme. FIG. 20B illustrates a multiplexed orthogonal system in which the primary crRNA/Cas enzyme is a Cas13a system, and the secondary crRNA is designed to be unblocked by the Cas13a enzyme, but the secondary crRNA is a Cas12 crRNA that complexes with a Cas12 enzyme to cleave a probe cleavable by a Cas 12 enzyme. FIG. 20C, illustrates the opposite, in which the primary system is a Cas12a system, and the secondary Cas enzyme is a Cas13a enzyme, with the secondary crRNA designed to complex with the Cas12a enzyme and the probe configured to be cleaved by the activated secondary Cas12a crRNA/Cas 12 system.

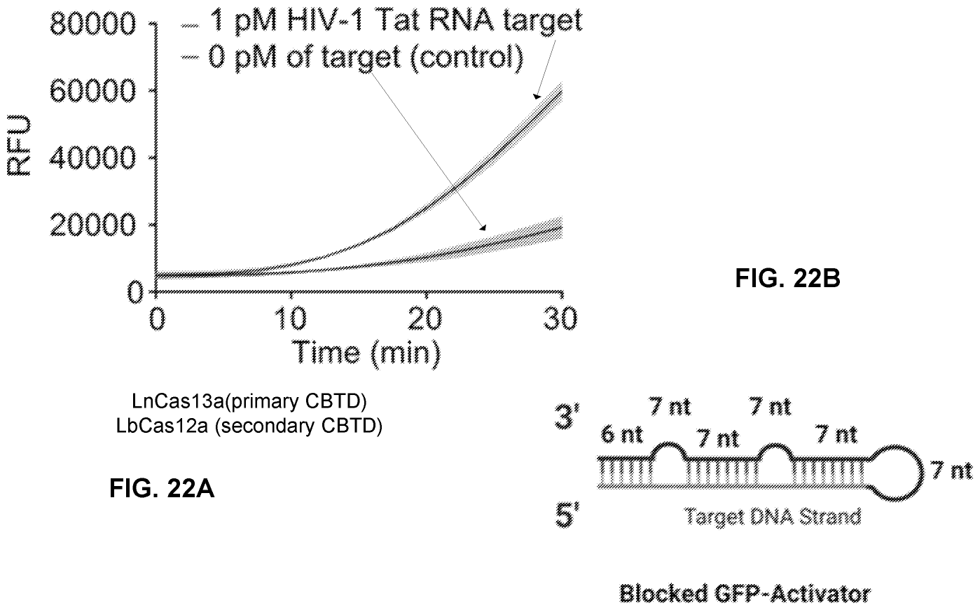

In this example, a multiplexed orthogonal system was tested having a Cas13a primary system and a Cas12a secondary system, such as illustrated in FIG. 20B. Different dilutions of a HIV Tat RNA were detected using CCR assay including Lne Cas13a as the primary Cas system and LbCas12a as the secondary Cas system. The assay used the hairpin blocked crGFP-14-3′ as the secondary crRNA as well as a secondary GFP activator blocked using a hairpin modified RNA blocker similar to the diagram shown in FIG. 19, shown in greater detail in FIG. 22B.

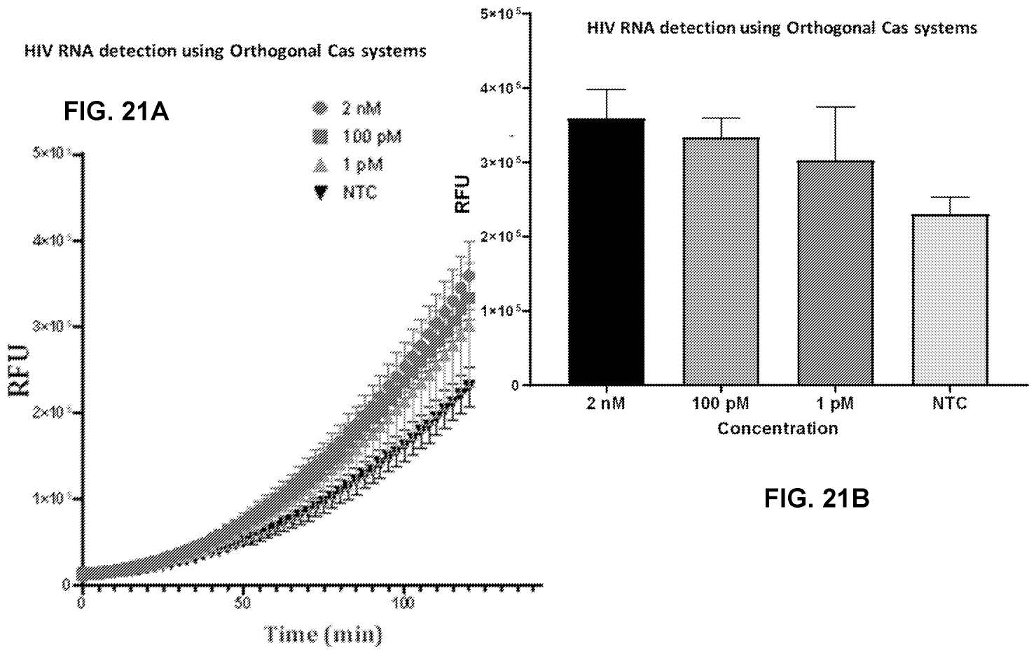

Several dilutions of a synthetic HIV RNA ranging from 2 nM-1 pM were detected using an orthogonal CCR system including LneCas13a as the primary system and LbCas12a as the secondary system. 50 nM each of crHIV, LneCas13a, LbCas12a and the hairpin modified crGFP-14-3′ were incubated with 5 nM of a single-stranded GFP-activator that is blocked with an RNA blocker through a hairpin extension at its 3′-end. 500 nM of FAM-FQ reporter was used. Fluorescence measurements were done every 2.5 mins at 37° C. using a Synergy Neo microplate reader. FIG. 21A shows a plot of RFU vs time at different concentrations (n=3). FIG. 21B shows mean RFU for 3 replicates at t=120 min is indicated. Error bars represent SD. NTC=No Target Control

LneCas13a (50 nM), crRNA (50 nM) were incubated for 10 min with cleavage buffer (2 mM HEPES-Na, 5 mM KCl, 0.5 mM MgCl2, 0.5% glycerol pH=6.8) at 37° C., and then the primary target of a 730 bases HIV-1 Tat RNA (1 nM) and the blocked secondary activator ssDNA shown in panel FIG. 22B (5 nM) were added to the mix. LbCas12a (50 nM), blocked secondary crRNA, ‘crGFP-14-3’ (50 nM) were combined in NEB 2.1 buffer reporter (500 nM) and then was added to the mix containing the primary CBTD system. The data shown in FIG. 22A shows that Cas13 and Cas12 can both be combined in a single assay for the detection of low copies of DNA or RNA using CCR. Mean±SE (n=3) are indicated in the graph.

### REFERENCES

1. Gootenberg J S, Abudayyeh O O, Lee J W, Essletzbichler P, Dy A J, Joung J, Verdine V, Donghia N, Daringer N M, Freije C A, Myhrvold C, Bhattacharyya R P, Livny J, Regev A, Koonin E V, Hung D T, Sabeti P C, Collins J J, Zhang F. Nucleic acid detection with CRISPR-Cas13a/C2c2. Science. 2017 Apr. 28; 356(6336):438-442. doi: 10.1126/science.aam9321. Epub 2017 Apr. 13. PMID: 28408723; PMCID: PMC5526198.
2. Chen, J. S., Ma, E., Harrington, L. B., Da Costa, M., Tian, X., Palefsky, J. M., & Doudna, J. A. (2018). CRISPR-Cas12a target binding unleashes indiscriminate single-stranded DNase activity. Science (New York, N. Y.), 360(6387), 436-439. https://doi.org/10.1126/science.aar6245
3. Shi K, Xie S, Tian R, et al., A CHRISPR-Cas autocatalysis-driven feedback amplification network for supersensitive DNA diagnostics. Sci Adv. 2021; 7(5); e-abc7802. Published 2021 Jan. 27. Dio: 10.1126/sciad.abc7802
4. Wang F, Wang L, Zou X, et al. Advances in CRISPR-Cas systems for RNA targeting, tracking and editing. Biotechnol Adv. March 2019; doi:10.1016/j.biotechadv.2019.03.016
5. Li Y, Li S, Wang J, Liu G. CRISPR/Cas Systems towards Next-Generation Biosensing. Trends Biotechnol. July 2019; 37(7):730-743. doi:10.1016/j.tibtech.2018.12.005
6. Xu X, Qi L S. A CRISPR-dCas Toolbox for Genetic Engineering and Synthetic Biology. J Mol Biol. January 2019; 431(1):34-47. doi:10.1016/j.jmb.2018.06.037
7. Yang B, Yang L, Chen J. Development and Application of Base Editors. CRISPR J. April 2019; 2:91-104. doi:10.1089/crispr.2019.0001
8. Zhang F. Exploration of Microbial Diversity to Discover Novel Molecular Technologies. Keio J Med. 2019; 68(1):26. doi:10.2302/kjm.68-002-ABST

We claim:

1. A CRISPR/Cas chain reaction (CCR) system for amplifying the detection sensitivity of a primary CRISPR-based target detection (CBTD) system that comprises a plurality of CRISPR-associated (Cas) enzymes with activatable trans-cleavage activity and a plurality of primary CRISPR RNAs (crRNA) capable of forming a complex with one of the Cas enzymes to form a primary crRNA/Cas complex, each primary crRNA comprising a primary guide sequence configured to bind a target, such that binding of the primary crRNA/Cas complex to the target activates the trans-cleavage activity of the Cas enzyme to produce an activated primary crRNA/Cas complex, the CCR system comprising:
a plurality of secondary crRNAs, each capable of forming a complex with one of the Cas enzymes to form a secondary crRNA/Cas complex, each secondary crRNA comprising a secondary guide sequence configured to bind an activator;
a plurality of activators, each activator comprising an oligonucleotide element complementary to and configured to bind the secondary crRNA, such that binding of the secondary crRNA/Cas complex to the activator activates the trans-cleavage activity of the Cas enzyme to produce an activated secondary crRNA/Cas complex;
a plurality of blocking moieties bound either to the secondary crRNA or to the activator such that the bound blocking moiety prevents binding of the secondary crRNA to the Cas enzyme or prevents binding of the secondary crRNA/Cas complex to the activator, each blocking moiety comprising a cleavable sequence configured to be cleaved by an activated Cas enzyme of an activated primary crRNA/Cas complex or an activated secondary crRNA/Cas complex, such that cleavage of the cleavable sequence of the blocking moiety releases the blocking moiety allowing the secondary crRNA to bind another of the Cas enzymes and an activator to produce an activated secondary crRNA/Cas complex, resulting in a CRISPR/Cas chain reaction that produces additional activated secondary crRNA/Cas complexes; and
a plurality of probes, each probe comprising an oligonucleotide element labeled with a detectable label, wherein the probe is configured to be cleaved by any of the activated Cas enzymes of the activated primary crRNA/Cas complexes or the activated secondary crRNA/Cas complexes to generate a detectable signal or a detectable molecule, wherein the CCR system amplifies the detection sensitivity compared to the primary CBTD system alone.

2. The CCR system of claim 1, wherein the blocking moiety is selected from: a nucleotide sequence configured to bind to the secondary crRNA or the activator that prevents binding of the secondary crRNA to the activator; a nucleotide sequence configured to bind to the secondary crRNA that prevents binding of the secondary crRNA to the Cas enzyme; and a large molecule or a surface linked to the secondary crRNA or the activator by a linking sequence that contains the cleavable sequence, such that the large molecule or surface sterically hinders binding of the secondary crRNA to the activator.

3. The CCR system of claim 2, wherein the blocking moiety is a blocking nucleotide sequence bound to the secondary crRNA that prevents binding of the secondary crRNA to the activator, wherein the blocking nucleotide sequence comprises one or more complementary segments bound to the secondary crRNA and one or more non-complementary, unbound segment having a sequence cleavable by an activated Cas enzyme, such that cleavage of the one or more non-complementary segments of the blocking nucleotide by the activated Cas enzyme releases the secondary crRNA from the blocking nucleotide such that the secondary crRNA can bind the activator.

4. The CCR system of claim 3, wherein at least one non-complementary, unbound segment occurs between at least two complementary segments, such that the non-complementary segment forms a bulge in the blocking nucleotide sequence.

5. The CCR system of claim 2, wherein the blocking moiety is a large molecule selected from: a protein; a lipid; a sugar; a nucleic acid; another large macromolecule; or a small molecule interacting with a magnetic particle, a nanoparticle, a peptide, a lipid, a sugar, a nucleic acid, or large macromolecule.

6. The CCR system of any of claims 1-5, wherein the target is a target polynucleotide sequence selected from: a ssDNA, a dsDNA, a ssRNA, a methylated DNA, a methylated RNA, or a heteroduplex of RNA and DNA.

7. The CCR system of any of claims 1-6, wherein the Cas enzyme is selected from Type V or Type VI Cas enzymes.

8. The CCR system of claim 7, wherein the Cas enzyme is selected from a Cas12 enzyme, a Cas13 enzyme, or a Cas14 enzyme.

9. The CCR system of claim 8, wherein the target is a ssDNA, a dsDNA, or a DNA/RNA heteroduplex sequence, and wherein the Cas12 enzyme is selected from: Cas 12a, Cas12b, Cas12c, Cas12d, Cas12e, Cas12f, Cas12g, Cas12h, Cas12i, Cas12j1-Cas12j10, or Cas12k.

10. The CCR system of claim 8, wherein the target is an RNA sequence and the Cas enzyme is a Cas13 enzyme selected from: Cas13a, Cas13b, Cas13c, or Cas13d

11. The CCR system of any of claims 1-10, wherein the CCR system further comprises an excess of one or more of the following: the Cas enzyme, in addition to the Cas enzyme in the primary CBTD system; the primary crRNA, in addition to the primary crRNA in the primary CBTD system; the activators; the secondary crRNAs; and the probes.

12. The CCR system of any of claims 1-9 or 11, wherein the Cas enzyme is a DNA-targeting nuclease and wherein the cleavable sequence of the blocking moiety and the oligonucleotide element of the probe comprise a ssDNA sequence having at least 80% A and/or T.

13. The CCR system of any of claims 1-8 or 10-12, wherein the Cas enzyme is an RNA-targeting nuclease and wherein the cleavable sequence of the blocking moiety and the oligonucleotide element of the probe comprise a ssRNA having at least 80% A and/or U.

14. The CCR system of any of claims 1-13, wherein the primary crRNA, the secondary crRNA, or both, comprise a polynucleotide extension sequence linked to a 3′-end of the guide sequence, the extension sequence having 1-31 nucleotides.

15. A CRISPR/Cas chain reaction (CCR) system for amplifying the detection sensitivity of a primary CRISPR-based target detection (CBTD) system that comprises a plurality of CRISPR-associated (Cas) enzymes with activatable trans cleavage activity and a plurality of primary CRISPR RNAs (crRNA) each comprising a primary guide sequence configured to bind a target and activate the primary CBTD system such that the primary crRNA and Cas enzyme form a primary crRNA/Cas complex that upon binding the target activates the trans-cleavage activity of the Cas enzyme to produce an activated primary crRNA/Cas complex, the CCR system comprising:
a plurality of secondary crRNAs capable of forming a complex with one of the Cas enzymes to form a secondary crRNA/Cas complex, each secondary crRNA comprising a secondary guide sequence configured to bind an activator;
a blocking nucleotide sequence bound to the secondary crRNA that prevents binding of the secondary crRNA to the Cas enzyme or the activator, wherein the blocking nucleotide sequence comprises one or more complementary segments bound to the secondary crRNA and one or more non-complementary, unbound segment having a sequence cleavable by an activated Cas enzyme of an activated primary crRNA/Cas complex or an activated secondary crRNA/Cas complex, such that cleavage of the one or more non-complementary segments of the blocking nucleotide releases the secondary crRNA from the blocking nucleotide such that the secondary crRNA can bind the activator and another of the Cas enzymes;
a plurality of activators, each activator comprising an oligonucleotide element complementary to and configured to bind the secondary crRNA, such that binding of the secondary crRNA/Cas complex to the activator activates the trans-cleavage activity of the Cas enzyme to produce an activated secondary crRNA/Cas complex, resulting in a CRISPR/Cas chain reaction that produces additional activated secondary crRNA/Cas complexes; and
a plurality of probes, each probe comprising an oligonucleotide element labeled with a detectable label, wherein the probe is configured to be cleaved by the activated Cas enzymes of the activated primary crRNA/Cas complexes or the activated secondary crRNA/Cas complexes to generate a detectable signal or a detectable molecule, wherein the CCR system amplifies the detection sensitivity compared to the primary CBTD system alone.

16. The CCR system of claim 15, wherein the blocking nucleotide sequence is connected to the secondary crRNA by a linker having a first end linked to a 3′ end of the secondary guide sequence and a second end linked to the blocking nucleotide sequence, such that the secondary guide sequence, linker, and blocking nucleotide sequence form a hairpin conformation, the linker having a sequence cleavable by the activated Cas enzyme, such that cleavage of the linker and any unbound segments of the blocking nucleotide sequence releases the secondary crRNA from the blocking nucleotide sequence.

17. The CCR system of any of claims 15-16, wherein at least one of the non-complementary, unbound segments occurs between at least two complementary segments, such that the non-complementary segment forms a bulge in the blocking nucleotide sequence.

18. The CCR system of any of claims 15-17, wherein the one or more complementary segments are each about 3-41 nucleotides long, and wherein the one or more non-complementary, unbound segments are each about 2-40 nucleotides long.

19. A kit comprising the CCR system of any of claims 1-18 and a primary CBTD system, the primary CBTD system comprising: a plurality of CRISPR-associated (Cas) enzymes with activatable trans cleavage activity and a plurality of primary CRISPR RNAs (crRNA) capable of forming a complex with one of the Cas enzymes to form a primary crRNA/Cas complex, each primary crRNA comprising a primary guide sequence configured to bind a target, such that binding of the primary crRNA/Cas complex to the target activates the trans-cleavage activity of the Cas enzyme to produce an activated primary crRNA/Cas complex.

20. The kit of claim 19, wherein the primary CBTD system comprises a commercially available CBTD system.

21. The kit of claim 19, wherein the primary crRNA comprises a polynucleotide extension sequence linked to a 3′-end of the guide sequence, and the polynucleotide extension sequence comprises a ssDNA or ssRNA having 1-31 nucleotides.

22. The kit of any of claims 19-21, wherein if the Cas enzyme is a DNA-targeting nuclease, the polynucleotide extension sequence comprises a ssDNA having a sequence of at least 80% A and/or T, but if the Cas enzyme is an RNA-targeting nuclease, the polynucleotide extension sequence comprises a ssRNA having a sequence of at least 80% A and/or U.

23. The kit of any of claims 19-22, wherein the kit comprises: an excess of the Cas enzyme, an excess of the primary crRNA, and an excess of activators.

24. A method of amplifying the detection sensitivity of a primary CRISPR-based target detection (CBTD) system, the method comprising combining the primary CBTD system with a sample comprising a target to be detected and a CRISPR/Cas chain reaction (CCR) system of any of claims 1-18, wherein the signal generated by the CCR system is greater than a signal produced from the primary CBTD system alone in the same amount of time.

25. The method of claim 24, wherein the target is not pre-amplified before combining with the primary CBTD system and/or the CCR system.

26. The method of claim 24-25, wherein the sample, the primary CBTD system and the CCR system are combined in a one-pot reaction.

27. The method of any of claims 24-26, performed at a temperature of about 20° C. to 75° C.

28. The method of any of claims 24-26, performed at about room temperature.

29. The method of any of claims 24-28, wherein the signal to be detected is selected from the group consisting of: fluorescence, luminescence, a chemical signal, a magnetic signal, a colorimetric signal, other optical signals, pH changes, temperature changes, electrochemical signals, and combinations of these.

30. A multiplexed CRISPR-based target detection (CBTD) system comprising:
a primary CRISPR-based target detection (CBTD) system that comprises a plurality of primary CRISPR-associated (Cas) enzymes with activatable trans-cleavage activity and a plurality of primary CRISPR RNAs (crRNA) capable of forming a complex with one of the primary Cas enzymes to form a primary crRNA/Cas complex, each primary crRNA comprising a primary guide sequence configured to bind a target, such that binding of the primary crRNA/Cas complex to the target activates the trans-cleavage activity of the primary Cas enzyme to produce an activated primary crRNA/Cas complex; and
a secondary CBTD system that comprises:
a plurality of secondary Cas enzymes with activatable trans cleavage activity, wherein the secondary Cas enzyme is different from the primary Cas enzyme;
a plurality of secondary crRNAs capable of forming a complex with a secondary Cas enzyme to form a secondary crRNA/Cas complex, each comprising a secondary guide sequence configured to bind an activator;
a plurality of activators, each activator comprising an oligonucleotide element complementary to and configured to bind the secondary crRNA, such that binding of the secondary crRNA/Cas complex to the activator activates the trans-cleavage activity of the secondary Cas enzyme to produce an activated secondary crRNA/Cas complex;
a plurality of blocking moieties bound either to the secondary crRNA or to the activator such that the bound blocking moiety prevents binding of the secondary crRNA to the secondary Cas enzyme or prevents binding of the secondary crRNA/Cas complex to the activator, each blocking moiety comprising a cleavable sequence configured to be cleaved by an activated Cas enzyme of an activated primary crRNA/Cas complex, such that cleavage of the cleavable sequence of the blocking moiety releases the blocking moiety allowing the secondary crRNA to bind a secondary Cas enzymes and an activator to produce an activated secondary crRNA/Cas complex; and
a plurality of probes, each probe comprising an oligonucleotide element labeled with a detectable label, wherein the probe is configured to be cleaved by the activated secondary crRNA/Cas complex to generate a detectable signal or a detectable molecule.

31. The multiplexed CBTD system of claim 30, wherein the primary Cas enzyme is a Cas12 enzyme, the secondary Cas enzyme is a Cas13 enzyme, and the target to be detected is a ssDNA, dsDNA, or DNA/RNA heteroduplex sequence.

32. The multiplexed CBTD system of claim 30, wherein the primary Cas enzyme is a Cas13 enzyme and the secondary Cas enzyme is a Cas12 enzyme, and the target to be detected is an RNA sequence.

33. A CRISPR chain reaction (CCR) system comprising:
a secondary CRISPR/Cas system configured to become activated upon activation of a primary CBTD system that comprises a plurality of primary CRISPR RNAs (crRNA) having a primary guide sequence configured to bind a specific target and form a complex with the target and one of a plurality of CRISPR-associated (Cas) enzymes with activatable trans-cleavage activity to form an activated primary crRNA/Cas complex, wherein the plurality of Cas enzymes is part of the primary CBTD system, the CCR system, or both, the secondary CRISPR/Cas system comprising:
a plurality of secondary crRNAs, each capable of forming a complex with one of the plurality of Cas enzymes to form a secondary crRNA/Cas complex, each secondary crRNA comprising a secondary guide sequence configured to bind an activator;
a plurality of activators, each activator comprising an oligonucleotide element complementary to and configured to bind the secondary crRNA, such that binding of the secondary crRNA/Cas complex to the activator activates the trans-cleavage activity of the Cas enzyme to produce an activated secondary crRNA/Cas complex;
a plurality of blocking moieties bound either to the secondary crRNA or to the activator such that the bound blocking moiety prevents binding of the secondary crRNA to the Cas enzyme or prevents binding of the secondary crRNA/Cas complex to the activator, each blocking moiety comprising a cleavable sequence configured to be cleaved by an activated Cas enzyme of an activated primary crRNA/Cas complex or an activated secondary crRNA/Cas complex, such that cleavage of the cleavable sequence of the blocking moiety releases the blocking moiety allowing the secondary crRNA to bind another of the Cas enzymes and an activator to produce an activated secondary crRNA/Cas complex, resulting in a CRISPR chain reaction that produces additional activated secondary crRNA/Cas complexes,
wherein each of the activated secondary crRNA/Cas complexes and each of the activated primary crRNA/Cas complexes is capable of cleaving a plurality of probes included in the primary CBTD system, the CCR system, or both, each probe comprising an oligonucleotide element labeled with a detectable label, the oligonucleotide element configured to be cleaved by any of the activated primary crRNA/Cas complexes or the activated secondary crRNA/Cas complexes to generate a detectable signal or a detectable molecule, wherein the CCR system amplifies the detection sensitivity compared to the primary CBTD system alone.
